(19) United States
(12) Patent Application Publication
(10) Pub. No.: US 2008/0019909 A1
(43) Pub. Date: Jan. 24, 2008

(54) Modulation of Programmed Necrosis

(76) Inventors: Francis Ka-Ming Chan, Shrewsbury, MA (US); Michael Lenardo, Potomac, MD (US)

Correspondence Address: FISH & RICHARDSON P.C., PO BOX 1022, MINNEAPOLIS, MN, 55440-1022, US

(21) Appl. No.: 10/571,799

(22) PCT Filed: Sep. 17, 2004
(86) PCT No.: PCT/US04/30737

§ 371 Date: Jan. 29, 2007

Related U.S. Application Data
(60) Provisional application No. 60/503,926, filed on Sep. 17, 2003.

(51) Int. Cl. G01N 33/566; A61K 49/00; C12N 5/06; C12Q 1/02

(52) U.S. Cl. 424/9.2; 435/29; 435/375; 435/7.21

(57) ABSTRACT

Methods related to modulating programmed necrosis are described. The methods relate to modulation of proteins involved in programmed necrosis, e.g., tumor necrosis factor receptor (2) (TNFR-2), tumor necrosis factor receptor (1) (TNFR-1), and receptor-interacting protein (RIP).

### CROSS-REFERENCE TO RELATED APPLICATIONS

This application claims priority to U.S. Application Ser. No. 60/503,926, filed on Sep. 17, 2003, the contents of which are hereby incorporated by reference in their entirety.

### FEDERALLY SPONSORED RESEARCH OR DEVELOPMENT

The U.S. Government may have certain rights in this invention.

### TECHNICAL FIELD

This invention relates to cell death.

### BACKGROUND

Tumor necrosis factor (TNF) is a pleiotropic cytokine that mediates diverse biological responses ranging from inflammation to cell death. Its crucial role in immune homeostasis is illustrated by the many autoimmune diseases that are etiologically related to TNF dysfunction (O'Shea et al., 2002, Nature Rev. Immunol 2:37-45). TNF is also a critical pro-inflammatory cytokine in innate immunity and is important for protection against certain bacterial and viral infections (Benedict et al., 2002, Nat. Immunol. 3:1013-1018; Herbein et al., 2000, Proc. Soc. Exp. Biol. Med. 223:241-257; Homeff et al., 2002, Nat. Immunol. 3:1033-1040). TNF exerts its biological functions mainly through binding to its two cell surface receptors, p55/p60 tumor necrosis factor receptor 1 (TNFR-1) and p75/p80 tumor necrosis factor receptor 2 (TNFR-2).

Signaling of the pre-assembled TNFR-1 results in the displacement of the Silencer of Death Domain (SODD) protein and the recruitment of the death domain (DD)-containing TNF-R1-associated death domain protein (TRADD) adapter. Subsequent binding of TNFR-associated factor 2 (TRAF2) or the protein serine/threonine kinase RIP (receptor-interacting protein) is critical for TNF-induced JNK linase and NF-KB activation, respectively (Wallach et al., 1999, Annu. Rev. Immunol 17:331-367; Chan et al., 2000, Immunity 13:419-422). In addition, binding of FADD and caspase-8 or caspase-10 to TRADD can initiate the caspase cascade, which results ultimately in cell death by apoptosis. Although TNFR-2 does not directly engage the apoptotic machinery due to the lack of a cytoplasmic DD, it can enhance the cell death signal of TNFR-1 through TRAF2 degradation (Chan et al., 2000, Eur. J Immunol. 30:652-660). TRAF2 degradation may enhance the recruitment of FADD and RIP to TNFR-1 due to reduced steric hindrance, thereby leading to enhanced cell death upon TNF stimulation (Fotin-Mleczek et al., 2002, J. Cell Sci. 115:2757-2770). However, direct competition among FADD, RIP, and TRAF2 for TNFR-1 binding has not been previously tested and the mechanism by which TNFR-2 signaling enhances TNFR-1 mediated cell death remains unknown.

### SUMMARY

The application is based, in part, on the discovery that tumor necrosis factor receptor 2 (TNFR-2) is involved in programmed necrosis in a manner that is dependent on receptor-interacting protein (RIP). It has also been found that induction of programmed necrosis is suppressed during apoptosis because RIP is cleaved and inactivated during apoptosis by caspase-8. In addition, it is shown that induction of cell death in vaccinia infected cells requires TNF-induced RIP-dependent programmed necrosis. It is also disclosed herein that certain viral gene products, termed “FLIPs” (FLICE-like inhibitor proteins) are potent inhibitors of programmed necrosis. Thus, TNF-induced, RIP-dependent programmed necrosis is a mechanism for controlling viral infections. Accordingly, enhancing programmed necrosis can be a means of enhancing anti-viral responses. Furthermore, inhibition of programmed necrosis can be useful for improving the efficacy and decreasing undesirable effects of vaccines such as smallpox and other poxviruses. Furthermore, inhibition of programmed necrosis is useful for treating disorders associated with an undesirable inflammatory response such as rheumatoid arthritis, inflammatory bowel diseases, and septic shock.

The invention includes methods of determining whether a test compound can modulate programmed necrosis by (a) providing a test cell capable of undergoing programmed necrosis; (b) contacting the test cell with an inducer of programmed necrosis and a test compound, thereby providing a test sample; and (c) determining the effect of the test compound on TNF Receptor (TNFR)-2 expression or activity, Receptor-Interacting Protein (RIP) degradation, RIP recruitment to Tumor Necrosis Factor Receptor (TNFR-1), or TNFR-Associated Factor 2 (TRAF2) degradation; wherein a change in the effect in the test sample compared to the effect in a test cell contacted with an inducer of programmed necrosis in the absence of the test compound indicates that the test compound can modulate programmed necrosis.

In one embodiment, the inducer of programmed necrosis is a Tumor Necrosis Factor (TNF), a TNF-related apoptosis-inducing ligand (TRAIL), or a Fas ligand. In certain embodiments, the test compound increases programmed necrosis. Alternatively, in other embodiments, the test compound decreases programmed necrosis. The test compound can affect one or more of TNFR-2 expression or activity, RIP degradation, RIP recruitment to TNFR-1, or TRAF2 degradation. In various embodiments, the cell contains a recombinant TNFR-2, and the test compound specifically binds to TNFR-2 or RIP. In certain embodiments, the test compound is an antibody or fragment thereof, and the method can further include inhibiting caspase-8 expression or activity in the test cell, or the cell is caspase 8 -/-.

The invention also includes methods of modulating programmed necrosis in a cell by (a) providing a cell that can undergo programmed necrosis and is optionally exposed to an inducer of programmed necrosis and (b) contacting the cell with a compound that can modulate TNFR-2 expression or activity, RIP degradation, RIP recruitment to TNFR-1, or TRAF2 degradation, thereby modulating programmed necrosis. In one embodiment, the inducer of programmed necrosis is a TNF, a TRAIL, or a Fas ligand. In various embodiments, the compound increases or decreases programmed necrosis. In specific embodiments, the compound modulates one or more of TNFR-2 expression or activity, RIP degradation, RIP recruitment to TNFR-1, or TRAF2 degradation. In certain embodiments, the cell contains a recombinant TNFR-2. In various embodiments, the methods can further include contacting the cell with a compound that modulates the expression or activity of caspase-8. The compound can specifically bind to TNFR-2 or RIP, and can be in the form of an antibody. In certain embodiments, the cell can be within a subject, such as a mammal (dog, cat, horse, cow, pig, or goat) or a human patient. In another embodiment, the cell is a cultured cell.

In certain embodiments, the compound decreases degradation of RIP, thereby increasing programmed necrosis. The method can also further include contacting the cell with a second compound that can induce programmed necrosis, such as a TNF, a TRAIL, or a Pas ligand. In other embodiments, the compound increases degradation of RIP, thereby decreasing programmed necrosis. Alternatively, the compound can increase recruitment of RIP to TNFR-1, thereby increasing programmed necrosis. In addition to increasing recruitment of RIP to TNFR-1, the method can further include contacting the cell with a compound that induces programmed necrosis.

In another aspect, the invention also includes methods of decreasing an inflammatory response in a cell by (a) providing a cell that is susceptible to or undergoing an inflammatory response and (b) contacting the cell with a compound that decreases TNFR-2 expression or activity, increases RIP degradation, decreases RIP recruitment to TNFR-1, or decreases degradation of TRAF-2, in an amount sufficient to decrease an inflammatory response. In certain embodiments, the cell is within a subject. The compound can be administered to the subject as a vaccine. As another example, the subject may be at risk for or has rheumatoid arthritis, an inflammatory bowel disease, or septic shock.

In another aspect, the invention also includes a method of modulating cytotoxicity in a cell that is infected with a virus or is susceptible to a viral infection by (a) providing a cell that is infected with a virus or is susceptible to viral infection and (b) contacting the cell with a compound that affects TNFR-2 expression or activity, RIP degradation, RIP recruitment to TNFR-1, or TRAF2 degradation, in an amount sufficient to affect cytotoxicity in the cell. In one embodiment, cytotoxicity is increased by increasing TNFR-2 expression or activity, decreasing RIP degradation, increasing RIP recruitment to TNFR-1, or increasing TRAF2 degradation. Alternatively, in other embodiments, cytotoxicity is decreased by decreasing TNFR-2 expression or activity, increasing RIP degradation, decreasing RIP recruitment to TNFR-1, or decreasing TRAF2 degradation. In some embodiments, the cell is within a subject. In other embodiments, the cell is in culture.

In another embodiment, cytotoxicity in a cell during viral infection is decreased by contacting the cell with a compound that decreases TNFR-2 expression or activity in an amount sufficient to decrease cytotoxicity in the cell during viral infection.

The invention also covers compositions that include compounds that modulate programmed necrosis, such as geldanamycin, a kinase-inactive form of RIP (K45A), vaccinia virus SPI-2 protein, and MC159 protein (from poxvirus), which all suppress programmed necrosis. Other compounds, such as zVAD-fink, an apoptosis inhibitor, promote programmed necrosis.

“Programmed necrosis” or “programmed necrotic cell death” is a mechanism of cell death that is morphologically distinguishable from apoptotic cell death and requires activity of certain molecular components including RIP. Most cells undergoing programmed necrosis are Annexin V positive and propidium iodide (PI) positive at the same time. This can be compared to cells undergoing apoptosis that are positive for Annexin V, but are negative for PI. Cells undergoing programmed necrosis exhibit a rapid loss of plasma membrane and show little chromatin condensation (e.g., as compared to an apoptotic cell, which has extensive chromatin condensation). The process of programmed necrosis requires RIP activity and is caspase independent.

A “biologically active portion” of a target protein (e.g., a TNFR-2, RIP, or TNFR-1 protein) includes a fragment of a target protein that participates in an interaction between a target molecule and a non-target molecule (e.g., a molecule that is a binding partner). Biologically active portions of a target protein include peptides comprising amino acid sequences sufficiently homologous to or derived from the amino acid sequence of a target protein, e.g., the amino acid sequence of Genbank Accession number P20333 (TNFR-2), P19438 (TNFR-1), and NP_{—}003795 (RIP), containing fewer amino acids than the full-length target protein and exhibiting at least one activity of a target protein.

A molecule that “specifically binds” is a molecule that binds to a particular entity, e.g., a TNFR-2 polypeptide, RIP polypeptide, or TNFR-1 polypeptide, but which does not substantially recognize or bind to other molecules in a sample, e.g., a biological sample, which includes the particular entity, e.g., a TNFR-2 polypeptide, RIP polypeptide, or TNFR-1 polypeptide.

A “polypeptide” means a chain of amino acids regardless of length or post-translational modifications.

“Subject,” as used herein, can refer to a mammal, e.g., a human, or to an experimental animal (e.g., disease) model. The subject can be a non-human animal, e.g., a mouse, rat, cat, dog, guinea pig, horse, cow, pig, goat, or other domestic animal.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Other features and advantages of the invention will be apparent from the detailed description, drawings, and from the claims.

### DESCRIPTION OF DRAWINGS

FIG. 1A is a graph showing the results of experiments in which caspase-8 -/- Jurkat cells I9.2 or I9.2 cells overexpressing TNFR-2 (J3.2) were treated with different doses of rhTNFα (recombinant human tumor necrosis factor α) for 16 hours. Live and dead cells were determined using flow cytometry (FACS: fluorescence activated cell sorting) by propidium iodide (PI) exclusion.

FIG. 1B is a graph showing the results of experiments in which FADD -/- Jurkat cells with (I2.1) or without (I42) TNFR-2 were stimulated with the indicated concentrations of rhTNF for 16 hours. Cell death was analyzed by PI exclusion on FACS.

FIG. 1C is a bar graph showing the results of experiments in which caspase-8 -/- J3.2 cells were stimulated with the indicated antibodies (3 μg/ml each) or 10 ng/ml rhTNFα for 16 hours prior to cell loss determination by PI exclusion on FACS. AB225 and Mab225 are antibodies against TNFR-1 (R1). AB226 and Mab226 are agonist and antagonist antibodies against TNFR-2 (R2), respectively.

FIG. 1D is a graph showing the results of experiments in which TNFR-2-expressing wild-type (4E3), FADD -/- (I42), or caspase-8 -/- (J3.2) Jurkat cells were treated with increasing concentrations of rhTNFα for 16 hours and cell loss was analyzed as in FIG. 1A. TNFR-2 cell surface expression in all three lines was similar by FACS staining (see FIG. 8).

FIG. 1E is a bar graph showing the results of experiments in which TNFR-2+ FADD -/- cells (I42) were transiently transfected with pEGFP-N1 as a transfection marker along with pcDNA3 (vector), full-length FADD (FADD-FL), or the FADD death domain (DD) alone (FADD-DD). Cells were stimulated with, TNF or anti-Fas antibody (CH11) for 12 hours and cell loss was analyzed on the GFP+ cells by PI exclusion on FACS.

FIG. 1F is an electron micrograph of TNF-stimulated caspase-8 -/- J3.2 cells. Note the swelling of cellular organelles (white arrow) and the extensive loss of membrane integrity (black arrow).

FIG. 1G is an electron micrograph of anti-Fas treated wild-type 4E3 cells. Classical apoptosis as distinguished by chromatin condensation (white arrow) and the preservation of membrane integrity (black arrow) are indicated.

FIG. 2A is a bar graph showing the results of experiments in which TNFR-2+ wild-type 4E3 (WT) and RIP -/- cells (R1.1) were stimulated as indicated with 10 ng/ml rhTNFα, rhTNFα plus 50 mM z-Val-Ala-Asp-fluoromethyl ketone (zVAD-fink), rhTNFα plus 0.5 mM geldanamycin (GA), or rhTNFα with zVAD-fmk plus GA. Cells were harvested and analyzed by PI exclusion on FACS 16 hours post-stimulation and cell loss was calculated as described in Example 1 (infra). The inset shows the reduction in RIP protein level when 4E3 cells were treated with GA.

FIG. 2B is a bar graph showing the results of experiments in which caspase-8 -/- J3.2 cells pretreated with 0.5 mM geldanamycin (GA) or left untreated were stimulated with the indicated doses of rhTNF for 15 hours. Cell loss was determined as described in Example 1 (infra).

FIG. 2C is a bar graph showing the results of experiments in which kinase inactive versions of RIP (RIP-K45A), or of ASK1 (ASK1-K709R), or control vector pcDNA3 were co-transfected with pEGFP-N1 into caspase-8 -/-J3.2 cells. Cells were treated with 10 ng/ml rhTNFα for 16 hours and live GFP positive cells were counted using FACS analysis to determine specific cell loss of transfected cells. Results were representative of three independent experiments.

FIG. 3A is a bar graph showing the results of experiments in which TNFR-2+ wild-type type, FADD-/-, and caspase-8 -/- cells were pre-stimulated with agonistic TNFR-2 antibody or left untreated for five hours followed by stimulation of TNF for four hours. Cell death was determined by a combination of Annexin V and PI staining.

FIG. 3B is a set of reproductions of Western blots of the indicated proteins present in the TNFR-1 signaling complex and control whole cell lysates blots showing equivalent inputs of each protein. The Western blots show the results of experiments in which wild-type (WT) or TNFR-2-expressing 4E3 cells were pretreated with agonistic TNFR-2 antibody for six hours to induce TNFR-associated factor 2 (TRAF2) degradation prior to stimulation with 100 ng/ml of rhTNFα for five minutes (Chan et al., 2000, Eur. J. Immunol. 30:652-660). Lysates were pre-cleared with three rounds of washing with protein G agarose beads prior to immunoprecipitation with anti-TNFR-1 antibody. Western blots were performed with antibodies to TRADD and RIP as indicated. Reprobing the blots revealed a loss of TRAF2 in the cell lysates and no contaminating TNFR-2 in the immunocomplexes.

FIG. 3C is a set of reproductions of Western blots showing the results of experiments in which I42 cells (FADD -/-) or wild-type 4E3 cells (WT) were stimulated with 100 ng/ml of rhTNFα for the indicated amount of time. Inmunoprecipitations (IP) were performed with TNFR-1-specific antibody and the same membranes were probed in Western blots using antibodies to RIP, TRADD, and TRAF2 as indicated. The whole cell extracts (WCE) showed that equivalent amounts of each protein were present in the cell lysates.

FIG. 4A is a Western blot showing the results of experiments in which wild-type 4E3 cells (WT) and J3.2 cells (Casp-8 -/-) cells Were treated with various combinations of rhTNFα, Apo-1 (anti-Fas), and zVAD-fmk for six hours before whole cell lysates were harvested. Western blot analysis using a C-terminal specific RIP antibody showed that full-length RIP was cleaved into a smaller fragment (RIPc) in 4E3 cells in response to TNF and anti-Fas stimulation.

FIG. 4B is a bar graph showing the results of experiments in which TNFR-2+ RIP -/- cells were transfected with the indicated plasmids and stimulated with TNF. Cell loss was determined by PI exclusion on FACS. The results are representative of three experiments.

FIG. 4C is a bar graph showing the results of experiments in which TNFR-2+ RIP -/- cells were transfected with the indicated plasmids and stimulated with TNF in the presence of zVAD-fmk. Cell loss was determined by PI exclusion on FACS. The results are representative of three experiments.

FIG. 5A is a bar graph depicting the results of experiments in which caspase-8 -/- J3.2 cells infected with wild-type recombinant VV (WT VV), recombinant VV with a deletion in SPI-2 (ASPI-2 VV), recombinant VV with MC159 substituting the SPI-2 gene MC159 VV), or left uninfected for six hours. Cells were then treated with rhTNFα for 14 hours. Infection and cell death were monitored by FACS using anti-E3L staining and forward/side scatter, respectively. TUNEL (Terminal deoxynucleotidyl Transferase Biotin-dUTP Nick End Labeling) staining was performed to distinguish apoptosis from programmed necrosis. Results shown are representative of three independent experiments.

FIG. 5B is a bar graph depicting the results of experiments in which RIP -/- R1.1 cells were infected with wild-type recombinant VV (WT VV), recombinant VV with a deletion in SPI-2 (ΔSPI-2 VV), recombinant VV with MC159 substituting the SPI-2 gene (MC159 VV), or left uninfected for six hours. Cells were then treated with rhTNFα for 14 hours. Infection and cell death were monitored by FACS using anti-E3L staining and forward/side scatter, respectively. TUNEL staining was performed to distinguish apoptosis from programmed necrosis. Results shown are representative of three independent experiments.

FIG. 5C is a graph depicting the results of experiments in which C57BL/6 wild-type control or TNFR-2 -/- mice (10-12 weeks old) were infected with 10⁶ pfu of VV by intraperitoneal injection. Organs were harvested four days post-infection and viral titers were determined by Vero cell plaque assay as described in Example 1. The open and closed circles represent the wild-type control mice and TNFR-2 -/- mice, respectively. Each point represents one mouse. The mean pfu (expressed in logarithmic scale) in each group of four mice in each organ is indicated by the bars and at the top of the graph.

FIGS. 6A-C are a set of photomicrographs showing the histopathology of liver and spleen of VV-infected wild-type and TNFR-2 -/- mice. The liver sections are stained with hemotoxylin/eosin.

FIGS. 6D-F are photomicrographs of spleen sections of C57BL/6 and TNFR-2 -/-mice.

FIG. 7A is a bar graph showing the results of experiments in which caspase-8 -/- J3.2 cells were transfected with pEGFP-N1 (empty vector), MC159-GFP (MC159), E8-GFP (E8), K13-GFP (K13) or cFLIPs-GFP (cFLIPs). Cells were stimulated with 1 ng/ml rhTNF, 10 ng/ml of rhTNF, or left untreated for 20 hours. The negative cell loss in the MC159 and E8 samples represent the proliferation of the cells expressing the protective protein.

FIG. 7B is a bar graph showing the results of experiments in which caspase 8 -/- J3.2 cells were transfected with the indicated plasmids and cell loss was determined by PI exclusion on FACS after 12 hours stimulation with TNF. Results are representative of three experiments.

FIG. 7C is a reproduction of a Western blot depicting the result of an experiment in which 293T cells were transfected with HA-tagged RIP and an increasing amount of HA-MC159 as indicated. Immunoprecipitation (IP) was performed with antibody specific to RIP and Western blot (WB) was performed with antibody specific to the HA epitope tag.

FIG. 7D is a reproduction of a Western blot depicting the result of an experiment in which 293T cells were transfected with the indicated plasmids. Immunoprecipitation was performed using TNFR-i-specific antibody (top panel) and Western blot analysis was performed using HA-specific antibody. The bottom panel shows the expression of the plasmids in the whole cell extracts.

FIG. 8A is a plot from FACS analysis in which expression of TNFR-2 in caspase-8 -/-, FADD -/-, and RIP -/-Jurkat clones. TNFR-2 expression in caspase-8 -/- clones (a) J2.2, (b) J3.1, (c) J3.2, and (d) J3.6 is shown.

FIG. 8B is a plot from FACS analysis in which expression of TNFR-2 in FADD -/-clones (a) I10, (b) I19, (c) I23, and (d) I42 is shown.

FIG. 8C Expression of TNFR-2 in RIP -/-Jurkat clones (a) R1.1, (b) R23, (c) R45, and (d) R46.

The numbers in each histogram in FIGS. 8A-C represent the percentage of cells that are in the TNFR-2-positive gate. The normal and heavy lines represent TNFR-2 expression in the parental and stable lines respectively.

FIG. 8D is a bar graph depicting the results of experiments in which the response of TNFR-2 expressing FADD -/-Jurkat clones was compared to TNFR-2 negative, wild-type Jurkat cells (WT) and parental FADD -/-cells (I2.1). Cells were stimulated for 16 hours with 0.5 ng/ml rhTNFα and cell loss was determined by PI exclusion. The results showed that multiple TNFR-2+FADD -/- clones all exhibited increased response to TNF.

FIG. 8E is a bar graph depicting the results of experiments in which multiple RIP -/- clones expressing TNFR-2 were stimulated with 10 ng/ml rhTNFα for 16 hours and cell loss was determined as in (D). Wild-type 4E3 cells were included for comparison. The numbers beneath each bar represent the clone number. The results showed that RIP is required for the enhancing effect of TNFR-2 on TNF-induced death.

FIG. 8F is a bar graph depicting the results of experiments in which FADD -/- I42 cells and caspase-8 -/- J3.2 cells (both are TNFR-2+) were stimulated with TNF in the presence or absence of zVAD-fmk for 16 hours. Cell loss was determined by PI exclusion on FACS. The results showed that TNF-induced death in I42 and J3.2 cells was not blocked by caspase inhibition.

FIG. 8G is a reproduction of a Western blot analysis of caspase-8 cleavage in wild-type 4E3 cells.

FIG. 8H is a reproduction of a Western blot analysis of I42 FADD -/- cells stimulated with rhTNF or anti-Fas antibody (CH11). The uncleaved pro-enzyme doublet and the cleaved intermediates p42/p45 are shown.

### DETAILED DESCRIPTION

Tumor necrosis factor receptors (TNFRs), including TNFR-1, Fas, and TRAIL receptors are involved in apoptosis, but can also trigger an alternative form of cell death that is morphologically distinct from apoptosis. Because of the obligate requirement of distinct molecular components including the protein serine/threonine kinase RIP, this alternative form of cell death is referred to herein as “programmed necrosis.”

It is demonstrated herein that TNFR-2 facilitates programmed necrosis through TNFR-1. It was also found that TNFR-2 facilitates programmed necrosis by enhancing RIP recruitment to TNFR-1. In addition, RIP is cleaved (degraded) and inactivated by caspase-8 during apoptosis. Programmed necrosis becomes the dominant death response when caspases are inhibited, such as when apoptosis is inhibited during a viral infection. These data provide a molecular explanation as to why some cells undergo programmed necrosis when apoptosis is inhibited by tetrapeptide inhibitors of caspases or dominant-negative FADD (Vercammen et al., 1998, J. Exp. Med. 187:1477-1485; Vercammen et al., 1998, J. Exp. Med. 188:919-930; Li et al., 2000, J. Virol. 74:7470-7477; Kawahara et al., 1998, J. Cell. Biol. 143:1353-1360; Matsumura et al., 2000, J. Cell Biol. 151:1247-1256; Khwaja et al., 1999, J. Biol. Chem. 274:36817-36823; Denecker et al., 2001, Cell Mol. Life Sci. 58:356-370; Pelagi et al., 2000, Eur. Cytokine Netw. 11:580-588).

Using vaccinia virus (VV) infection as a model, it is shown herein that RIP-dependent programmed necrosis is essential for the in vitro killing of infected Jurkat cells infected with VV and TNFR-2 is required to control viral replication in vivo. Thus, increasing the expression or activity of molecules involved in programmed necrosis, such as RIP or TNFR-2, can increase programmed necrosis and viral killing. Decreasing the expression or activity of a molecule involved in programmed necrosis can decrease programmed necrosis and viral killing and ameliorate undesirable effects of programmed necrosis such as an inflammatory response.

Necrotic cell death causes an inflammatory signal that enhances the immune response. Dendritic cells (DC) are involved in antigen presentation to T cells and B cells. Although DC capture both necrotic and apoptotic cells, it is only necrotic cells that can activate DC to trigger lymphocyte activation. Therefore, compounds and methods that regulate the programmed necrosis pathway are useful for manipulating immune responses, for example, to enhance an immune response or to damp down an undesirably robust inflammatory reaction in response to challenge of the immune system (e.g., vaccination). TNFR-2 -/-mice exhibited a dramatic reduction in the number of inflammatory foci in the liver. Since programmed necrotic cell death is highly pro-inflammatory and an inflammatory reaction is critical for optimal priming of DC, the results disclosed herein suggest that programmed necrosis induced by TNF and other death cytokines may bolster adaptive immune response against viral infections by stimulating DC maturation and enhancing the uptake and presentation of viral antigens (Gallucci et al., 1999, Nat. Med. 5:1249-1255; Sauter et al., 2000, J. Exp. Med. 191:423-434). Thus, TNFR-2-facilitated programmed necrosis can be a crucial anti-viral response, especially for viruses that can block apoptosis. Accordingly, increasing TNFR-2 expression or activity can increase an antiviral response. Decreasing TNFR-2 expression or activity can be used to inhibit an undesirable inflammatory response, for example, in response to vaccination.

Cell Death Assays

The invention includes methods of identifying compounds that can modulate programmed necrosis. In some embodiments, cell death or necrosis is assayed. Methods for assaying necrotic cell death (programmed necrosis) are known in the art. Such methods include examination of cell morphology. For example, cells can be plated, washed with phosphate-buffered saline (PBS) then treated with trypan blue for one minute. Cells are then washed with PBS and examined under a microscope. Cells that exclude trypan blue and show membrane blebbing and apoptotic bodies are considered apoptotic, while cells that are trypan blue permeable and exhibit a balloon-like morphology are considered necrotic (Li et al., 2000, J. Virol. 74:7470-7477). Phosphatidylserine (PS) surface exposure is also increased early in the process of programmed necrosis and can be used as an assay of induction of programmed necrosis as described in Holler et al. (2000, Nature Immunol. 1:489-495). Superoxides are generated during necrotic cell death. Thus, assays for superoxides can be used to measure necrotic cell death. For example, dihydroethidium (DHE) binding can be assayed as described in Li et al. (2000, supra). An increase in DHE binding indicates an increase in programmed necrosis in a sample.

Screening Assays

The invention provides methods (also referred to herein as “screening assays”) for identifying modulators, i.e., candidate compounds or agents (e.g., proteins, peptides, peptidomimetics, peptoids, small molecules, oligonucleotides, siRNA, or other drugs) as described herein. For example, the assays can be used to identify compounds that modulate programmed necrosis (e.g., TNF-induced programmed necrosis). The assays can be used to identify compounds that modulate (increase or decrease) expression or activity of TNFR-2, compounds that modulate degradation of RIP, or compounds that modulate the recruitment of RIP to TNFR-1. Thus, the compounds can have a stimulatory or inhibitory effect on programmed necrosis. The identified compounds can be used to modulate programmed necrosis in a therapeutic protocol or to elaborate the mechanism of programmed necrosis. The actions of inhibitory agents include inhibition of TNFR-2 expression or activity, increasing RIP degradation, decreasing RIP kinase activity, or decreasing RIP recruitment to TNFR-1.

In one embodiment, the invention provides assays for screening candidate or test compounds that bind to TNFR-2 or RIP or a fragment thereof. In some embodiments, the assay identifies compounds that bind TNFR-1. Compounds that interfere with or enhance the binding between RIP and TNFR-1 can also be identified. In another embodiment, the invention provides assays for screening test compounds that modulate the expression or activity of a TNFR-2 or RIP (e.g., by inhibiting or enhancing degradation of RIP), or a biologically active portion thereof.

The test compounds of the present invention can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive (see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, K. S., 1997, Anticancer Drug Des. 12:145).

Examples of methods for the synthesis of molecular libraries can be found in the art, for example in: DeWitt et al., 1993, Proc. Natl. Acad. Sci. U.S.A. 90:6909; Erb et al., 1994, Proc. Natl. Acad. Sci. USA 91:11422; Zuckermann et al., 1994, supra.; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al,. 1994, J. Med. Chem. 37:1233.

Libraries of compounds may be presented in solution (e.g., Houghten, 1992, Biotechniques 13:412-421), on beads (Lam, 1991, Nature 354:82-84), chips (Fodor, 1993, Nature 364:555-556), bacteria (Ladner et al., U.S. Pat. No. 5,223,409), spores (Ladner et al., U.S. Pat. No. 5,223,409), plasmids (Cull et al., 1992, Proc. Natl. Acad. Sci. USA 89:1865-1869) or on phage (Scott and Smith, 1990, Science 249:386-390; Devlin, 1990, Science 249:404-406; Cwirla et al., 1990, Proc. Natl. Acad. Sci. 87:6378-6382; Felici, 1991, J. Mol. Biol. 222:301-310; Ladner et al., supra.).

The assay can be a cell-based assay in which a cell that expresses a target (TNFR-2, RIP, or TNFR-1 protein or biologically active portion thereof) is contacted with a test compound and the ability of the test compound to modulate activity of the target is determined. Determining the ability of the test compound to modulate target activity can be accomplished by monitoring, for example, RIP activity. Such activity can be assayed as described in the art by assaying the phosphorylation of cellular proteins using orthophosphate labeling. In general, a RIP activity assay is used in a whole cell assay in which the object is to determine whether the test compound modulates the rate of degradation of RIP, e.g., in the presence of TNF or in the presence of apoptotic signaling. In the latter case, it is particularly desirable to identify compounds that decrease the amount of RIP degradation in the presence of the apoptotic signal.

The ability of the test compound to modulate target binding to a compound, e.g., binding of RIP and TNFR-1, can also be evaluated. This can be accomplished, for example, by coupling the compound, e.g., the substrate, with a radioisotope or enzymatic label such that binding of the compound, e.g., the substrate, to the target can be determined by detecting the labeled compound, e.g., substrate, in a complex. Alternatively, the target can be coupled with a radioisotope or enzymatic label to monitor the ability of a test compound to modulate target binding to a substrate in a complex. For example, compounds can be labeled with ¹²⁵I ³⁵S, ¹⁴C, or ³H, either directly or indirectly, and the radioisotope detected by direct counting of radio emission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound to interact with a target with or without the labeling of any of the interactants can be evaluated. For example, a microphysiometer can be used to detect the interaction of a compound with TNFR-2 or RIP without the labeling of either the compound or the target (McConnell, H. M. et al., 1992, Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and a target.

A cell-free assay is also provided in which a target protein (e.g., TNFR-2, RIP, or TNFR-1) or a biologically active portion thereof is contacted with a test compound and the ability of the test compound to bind to the target protein or the biologically active portion thereof is evaluated. The biologically active portions of the target proteins to be used in assays of the present invention generally include fragments that participate in interactions with non-target molecules, e.g., fragments with high surface probability scores.

Soluble and/or membrane-bound forms of isolated proteins (e.g., TNFR-2, TNFR-1, or RIP proteins or biologically active portions thereof) can be used in cell-free assays. When membrane-bound forms of the protein (e.g., TNRF-2) are used, it may be desirable to utilize a solubilizing agent. Examples of such solubilizing agents include non-ionic detergents such as n-octylglucoside, n-dodecylglucoside, n-dodecyhnaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

Cell-free assays involve preparing a reaction mixture of the target protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169; Stavrianopoulos, et al., U.S. Pat. No. 4,868,103). A fluorophore label on the first, “donor” molecule is selected such that its emitted fluorescent energy will be absorbed by a fluorescent label on a second, “acceptor” molecule, which in turn is able to fluoresce due to the absorbed energy. Alternately, the “donor” protein molecule may simply utilize the natural fluorescent energy of tryptophan residues. Labels are chosen that emit different wavelengths of light, such that the ‘acceptor’ molecule label may be differentiated from that of the “donor”. Since the efficiency of energy transfer between the labels is related to the distance separating the molecules, the spatial relationship between the molecules can be assessed. In a situation in which binding occurs between the molecules, the fluorescent emission of the “acceptor” molecule label in the assay should be maximal. An FET binding event can be conveniently measured through standard fluorometric detection means well known in the art (e.g., using a fluorimeter).

In another embodiment, determining the ability of a protein to bind to another molecule (e.g., a test compound) can be accomplished using real-time Biomolecular Interaction Analysis (BIA) (see, e.g., Sjolander, S. and Urbaniczky, C,. 1991, Anal. Chem. 63:2338-2345 and Szabo et al., 1995, Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between molecules.

In one embodiment, the target protein (e.g., TNFR-2, TNFR-1, or RIP, or a biologicaly active portion thereof) or the test compound is anchored onto a solid phase. The test compound complexes anchored on the solid phase can be detected at the end of the reaction. In general, the target can be anchored onto a solid surface, and the test compound, (which is not anchored), can be labeled, either directly or indirectly, with detectable labels discussed herein.

It maybe desirable to immobilize either TNFR-2, TNFR-1, RIP, or a biologically active portion thereof, or an antibody that specifically binds a target to facilitate separation of complexed from uncomplexed forms of one or both of the proteins, as well as to accommodate automation of the assay. Binding of a test compound to a target, interaction of an RIP with TNFR-1, or other protein interaction described herein in the presence and absence of a test compound can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase -TNFR-2, -TNFR-1, or -RIP fusion proteins can be adsorbed onto glutathione Sepharose beads (Sigma Chemical, St. Louis, Mo.) or glutathione-derivatized microtiter plates, which are then combined with the test compound or the test compound and either the non-adsorbed target, and the mixture incubated under conditions conducive to complex formation (e.g., at physiological conditions for salt and pH). Following incubation, the beads or microtiter plate wells are washed to remove any unbound components, the matrix immobilized in the case of beads, and complex determined either directly or indirectly, for example, as described above. Alternatively, the complexes can be dissociated from the matrix and the level of target binding or activity determined using standard techniques.

Other techniques for immobilizing either a target protein or a target molecule on matrices include using conjugation of biotin and streptavidin. Biotinylated target protein or target molecules can be prepared from biotin-NHS (N-hydroxy-succinimide) using techniques known in the art (e.g., biotinylation kit, Pierce Chemicals, Rockford, IL), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies that specifically bind to a target protein or target molecules but which do not interfere with binding of the protein to its binding partner (e.g., binding of RIP to TNFR-1). Such antibodies can be derivatized to the wells of the plate, and unbound binding partner or target protein trapped in the wells by antibody conjugation. Methods for detecting such complexes, in addition to those described above for the GST-immobilized complexes, include immunodetection of complexes using antibodies reactive with the target protein or binding partner, as well as enzyme-linked assays which rely on detecting an enzymatic activity associated with the target protein or binding partner.

Alternatively, cell-free assays can be conducted in a liquid phase. In such an assay, the reaction products are separated from unreacted components by any of a number of known techniques, including but not limited to: differential centrifugation (see, for example, Rivas, G., and Minton, A. P., 1993, Trends Biochem. Sci. 18:284-287); chromatography (gel filtration chromatography, ion-exchange chromatography); electrophoresis (see, e.g., Ausubel, F. et al., eds. Current Protocols in Molecular Biology 1999, J. Wiley: New York.); and immunoprecipitation (see, for example, Ausubel, F. et al., eds., 1999, Current Protocols in Molecular Biology, J. Wiley: New York). Such resins and chromatographic techniques are known to one skilled in the art (see, e.g., Heegaard, N. H., 1998, J. Mol. Recognit. 11: 141-8; Hage, D. S. and Tweed, S. A., 1997, J. Chromatogr. B. Biomed. Sci. Appl. 699:499-525). Further, fluorescence energy transfer may also be conveniently utilized, as described herein, to detect binding without further purification of the complex from solution.

In certain embodiments, the assay includes contacting the target protein or a biologically active portion thereof with a known compound that binds the target (e.g., RIP and TNFR-1) to form an assay mixture, contacting the assay mixture with a test compound, and determining the ability of the test compound to interact with a target protein, wherein determining the ability of the test compound to interact with a target protein includes determining the ability of the test compound to preferentially bind to the target or a biologically active portion thereof, or to modulate the activity of a target, as compared to the known compound.

The target proteins described herein can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the targetprotein. Such compounds can include, but are not limited to, molecules such as antibodies, peptides, and small molecules (e.g., small non-nucleic acid organic molecules or small inorganic molecules). In some cases, the assay provides methods for determining the ability of the test compound to modulate the activity of a target through modulation of the activity of a downstream effector of the target. For example, a test compound that can interact with TNFR-2 can be assayed for its ability to increase or decrease programmed necrosis.

To identify compounds that interfere with the interaction between a target and its cellular or extracellular binding partner(s), a reaction mixture containing the target and the binding partner is prepared, under conditions and for a time sufficient, to allow the two products to form a complex. To test an inhibitory agent, the reaction mixture is provided in the presence and in the absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of complexes between the target and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products. A reference can be established by averaging control reactions and can be used for comparing the assay performed in the presence of the test compound, thus limiting the need to perform a control for every assay.

These assays can be conducted in a heterogeneous or homogeneous format. Heterogeneous assays involve anchoring either the target or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface. To conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected by employing a solid phase format. For example, an immobilized antibody specific for one of the binding components to anchor any complexes formed in solution, and a labeled antibody specific for the other partner to detect anchored complexes. Depending upon the order of addition of reactants to the liquid phase, test compounds that inhibit complex or that disrupt preformed complexes can be identified.

In an alternate embodiment of the invention, a homogeneous assay can be used. For example, a preformed complex of the target and the interactive cellular or extracellular binding partner product is prepared in that either the target or its binding partner is labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target-binding partner interaction can be identified.

In yet another aspect, a target protein and its binding partner can be used in a two-hybrid assay or three-hybrid assay (see, e.g., Saifer et al., U.S. Pat. No. 5,283,317; Zervos et al., 1993, Cell 72:223-232; Madura et al., 1993, J. Biol. Chem. 268:12046-12054; Bartel et al., 1993, Biotechniques 14:920-924; Iwabuchi et al., 1993, Oncogene 8:1693-1696; and Brent WO94/10300). In such assays, compounds that modulate the interaction between a target and its binding partner can identified.

The two-hybrid system is based on the modular nature of most transcription factors, which consist of separable DNA-binding and activation domains. Briefly, the assay utilizes two different DNA constructs. In one construct, the gene that codes for a target protein (e.g., RIP) is fused to a gene encoding the DNA binding domain of a known transcription factor (e.g., GAL-4). In the other construct, a DNA sequence coding for a binding partner of the target (e.g., TNFR-1) is fused to a gene that codes for the activation domain of the known transcription factor. If the target and binding partner proteins are able to interact, in vivo, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected. A test compound can be added to such an assay and the ability of the test compound to increase or decrease transcription of the reporter gene indicates the ability of the test compound to increase or decrease the interaction between the target and binding partner.

In another embodiment, modulators of target expression are identified. For example, a cell or cell-free mixture is contacted with a test compound and the expression of target MRNA or protein is evaluated relative to the level of expression of target MRNA or protein in the absence of the test compound. When expression of target MRNA or protein is greater in the presence of the candidate compound than in its absence, the test compound is identified as a stimulator of target MRNA or protein expression. Alternatively, when expression of target MRNA or protein is less (statistically significantly less) in the presence of the test compound than in its absence, the test compound is identified as an inhibitor of target MRNA or protein expression. The level of target MRNA or protein expression can be determined by methods described herein for detecting target MRNA or protein.

In another aspect, the invention pertains to a combination of two or more of the assays described herein. For example, a modulating agent can be identified using a cell-based or a cell-free assay, and the ability of the agent to modulate the activity of a target protein can be confirmed in vivo, e.g., in an animal infected with a virus such as vaccinia.

This invention further pertains to novel agents identified by the above-described screening assays. Accordingly, it is within the scope of this invention to further use an agent identified as described herein (e.g., an agent that can modulate TNFR-2 expression or activity, an agent that can modulate RIP degradation, or an agent that can modulate the recruitment of RIP by TNFR-1) in an appropriate animal model to determine the efficacy of treatment with such an agent, toxicity, side effects, or mechanism of action. Furthermore, novel agents identified by the above-described screening assays can be used for treatments as described herein.

Animal models, e.g., of viral infection, are known in the art and are described herein. Such models can also be used in assay methods as described herein to identify compounds that modulate the expression or activity of TNFR-2, RIP, or TNFR-1. Such models can also be used to determine the effects of such compounds on, e.g., an inflammatory response, in vivo cytotoxicity, modulation of viral load, severity of infection, or duration of infection. Methods of identifying such conditions are known in the art.

Compounds that can be used in the assays described herein and that can be useful as pharmaceutical compositions also include siRNA, ribozymes, and antisense oligonucleotides. Methods of malking such compounds are known in the art and are described below.

RNA Interference

RNA interference (RNAi) is an efficient process whereby double-stranded RNA (dsRNA, also referred to herein as siRNAs (small interfering RNAs) or ds siRNAs, for double-stranded small interfering RNAs,) induces the sequence-specific degradation of homologous mRNA in animals and plant cells (Hutvagner and Zamore, 2002, Curr. Opin. Genet. Dev.:12,225-232; Sharp, 2001, Genes Dev., 15:485-490). In mammalian cells, RNAi can be triggered by 21-nucleotide (nt) duplexes of siRNA (Chiu et al., 2002, Mol. Cell. 10:549-561; Elbashir et al., 2001, Nature 411:494-498), by micro-RNAs (miRNA), functional small-hairpin RNA (shRNA), or other dsRNAs which are expressed in vivo using DNA templates with RNA polymerase III promoters (Zeng et al., 2002, Mol. Cell 9:1327-1333; Paddison et al., 2002, Genes Dev. 16:948-958; Lee et al., 2002, Nature Biotechnol. 20:500-505; Paul et al., 2002, Nature Biotechnol. 20:505-508; Tuschl, 2002,Nature Biotechnol. 20:440-448; Yu et al., 2002, Proc. Natl. Acad. Sci. USA 99(9):6047-6052; McManus et al., 2002, RNA 8:842-850; Sui et al., 2002, Proc. Natl. Acad. Sci. USA 99(6):5515-5520).

Accordingly, the invention includes such molecules that are targeted to a TNFR-2, TNFR-1, or RIP RNA. Molecules that can decrease the amount of TNFR-2 mRNA or a RIP mRNA are useful for decreasing or preventing programmed necrosis. Molecules that can decrease the amount of a TNFR-1 RNA are useful for increasing programmed necrosis.

siRNA Molecules

The nucleic acid molecules or constructs of the invention include dsRNA molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is identical or substantially identical to the first strand. The dsRNA molecules of the invention can be chemically synthesized or can be transcribed in vitro from a DNA template, or in vivo from, e.g., shRNA. The dsRNA molecules can be designed using any method known in the art, for instance, by using the following protocol:

1. Beginning with the AUG start codon, look for AA dinucleotide sequences; each AA and the 3′ adjacent 16 or more nucleotides are potential siRNA targets. siRNAs taken from the 5′ untranslated regions (UTRs) and regions near the start codon (within about 75 bases or so) may be less useful as they may be richer in regulatory protein binding sites, and UTR-binding proteins and/or translation initiation complexes may interfere with binding of the siRNP or RISC endonuclease complex. Thus, in one embodiment, the nucleic acid molecules are selected from a region of the cDNA sequence beginning 50 to 100 nucleotides downstream of the start codon. Further, siRNAs with lower G/C content (35-55%) may be more active than those with G/C content higher than 55%. Thus in one embodiment, the invention includes nucleic acid molecules having 35-55% G/C content. In addition, the strands of the siRNA can be paired in such a way as to have a 3′ overhang of 1 to 4, e.g., 2, nucleotides. Thus in another embodiment, the nucleic acid molecules can have a 3′ overhang of 2 nucleotides, such as TT. The overhanging nucleotides can be either RNA or DNA.

2. Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at www.ncbi.nlm.nih.gov/BLAST.

3. Select one or more sequences that meet required criteria for evaluation. Further general information about the design and use of siRNA can be found in “The siRNA User Guide,” available at mpibpc.gwdg.de/abteilungen/100/105/sirna.html.

Negative control siRNAs should have the same nucleotide composition as the selected siRNA, but without significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

The nucleic acid compositions of the invention include both siRNA and crosslinked siRNA derivatives. Crosslinking can be employed to alter the phannacokinetics of the composition, for example, to increase half-life in the body. Thus, the invention includes siRNA derivatives that include siRNA having two complementary strands of nucleic acid, such that the two strands are crosslinked. For example, a 3′ OH terminus of one of the strands can be modified, or the two strands can be crosslinked and modified at the 3′OH terminus. The siRNA derivative can contain a single crosslink (e.g., a psoralen crosslink). In some embodiments, the siRNA derivative has at its 3′ terminus a biotin molecule (e.g., a photocleavable biotin), a peptide (e.g., a Tat peptide), a nanoparticle, a peptidomimetic, an organic compound (e.g., a dye such as a fluorescent dye), or dendrimer. Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The nucleic acid compositions of the invention can be unconjugated or can be conjugated to another moiety, such as a nanoparticle, to enhance a property of the compositions, e.g., a pharmacokinetic parameter such as absorption, efficacy, bioavailability, and/or half-life. The conjugation can be accomplished by methods known in the art, e.g., using the methods of Lambert et al. (2002, Drug Deliv. Rev.:47(1), 99-112; describing nucleic acids loaded to polyalkylcyanoacrylate (PACA) nanoparticles); Fattal et al. (1998, J. Control. Release 53(1-3):137-43; describing nucleic acids bound to nanoparticles); Schwab et al. (1994, Ann. Oncol. 5 Suppl. 4:55-8; describing nucleic acids linked to intercalating agents, hydrophobic groups, polycations, or PACA nanoparticles); and Godard et al. (1995, Eur. J. Biochem. 232(2):404-410; describing nucleic acids linked to nanoparticles).

The nucleic acid molecules of the present invention can also be labeled using any method known in the art; for instance, the nucleic acid compositions can be labeled with a fluorophore, e.g., Cy3, fluorescein, or rhodamine. The labeling can be carried out using a kit, e.g., the SILENCER™ siRNA labeling kit (Ambion). Additionally, the siRNA can be radiolabeled, e.g., using ³H, ³²P, or other appropriate isotope.

siRNA Delivery for Longer-Term Expression

Synthetic siRNAs can be delivered into cells by cationic liposome transfection and electroporation. However, these exogenous siRNAs show only short term persistence of the silencing effect (generally about 4-5 days). Several strategies for expressing siRNA duplexes within cells from recombinant DNA constructs allow longer-term target gene suppression in cells, including mammalian Pol III promoter systems (e.g., H1 or U6/snRNA promoter systems (Tuschl, 2002, supra) capable of expressing functional double-stranded siRNAs; (Bagella et al., 1998, J. Cell. Physiol. 177:206-213; Lee et al., 2002, supra; Paul et al., 2002, supra; Yu et al., 2002, supra; Sui et al., 2002, supra). Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by an H1 or U6 snRNA promoter and expressed in cells, can inhibit target gene expression (Bagella et al., 1998, supra; Lee et al., 2002, supra; Paul et al., 2002, supra; Yu et al., 2002, supra; Sui et al., 2002, supra). Constructs containing siRNA sequence under the control of T7 promoter also make functional siRNAs when cotransfected into the cells with a vector expression T7 RNA polymerase.

Animal cells express a range of noncoding RNAs of approximately 22 nucleotides termed micro RNA (miRNAs) and can regulate gene expression at the post transcriptional or translational level during animal development. One common feature of miRNAs is that they are all excised from an approximately 70 nucleotide precursor RNA stem-loop, probably by Dicer, an RNase III-type enzyme, or a homolog thereof. By substituting the stem sequences of the miRNA precursor with miRNA sequence complementary to the target mRNA, a vector construct that expresses the novel miRNA can be used to produce siRNAs to initiate RNAi against specific mRNA targets in mammalian cells (Zeng, 2002, supra). When expressed by DNA vectors containing polymerase III promoters, micro-RNA designed hairpins can silence gene expression (McManus, 2002, supra). Viral-mediated delivery mechanisms can also be used to induce specific silencing of targeted genes through expression of siRNA, for example, by generating recombinant adenoviruses harboring siRNA under RNA Pol II promoter transcription control. Infection of HeLa cells by these recombinant adenoviruses allows for diminished endogenous target gene expression. Injection of the recombinant adenovirus vectors into transgenic mice expressing the target genes of the siRNA results in in vivo reduction of target gene expression. In an animal model, whole-embryo electroporation can efficiently deliver synthetic siRNA into post-implantation mouse embryos (Calegari et al., 2002, Proc. Natl. Acad. Sci. USA 99(22):14236-40). In adult mice, efficient delivery of siRNA can be accomplished by “high-pressure” delivery technique, a rapid injection (within 5 seconds) of a large volume of siRNA containing solution into animal via the tail vein (Lewis, 2002, Nature Genetics 32:107-108). Nanoparticles and liposomes can also be used to deliver siRNA into animals.

Uses of Engineered RNA Precursors to Induce RNAi

Engineered RNA precursors, introduced into cells or whole organisms as described herein, will lead to the production of a desired siRNA molecule. Such an siRNA molecule will then associate with endogenous protein components of the RNAi pathway to bind to and target a specific MRNA sequence for cleavage and destruction. In this fashion, the MRNA to be targeted by the siRNA generated from the engineered RNA precursor will be depleted from the cell or organism, leading to a decrease in the concentration of the protein encoded by that mRNA in the cell or organism.

Antisense

An “antisense” nucleic acid can include a nucleotide sequence that is complementary to a “sense” nucleic acid encoding a protein, e.g., complementary to the coding strand of a double-stranded cDNA molecule or complementary to a target mRNA sequence, e.g., a TNFR-2, TNFR-1, or RIP MRNA sequence.

An antisense nucleic acid can be designed such that it is complementary to the entire coding region of a target MRNA, e.g., a TNFR-2, TNFR-1, or RIP MRNA, but can also be an oligonucleotide that is antisense to only a portion of the coding or noncoding region (e.g., the 5′ or 3′ untranslated regions) of the target MRNA. For example, the antisense oligonucleotide can be complementary to the region surrounding the translation start site of the target mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The new antisense nucleic acid molecules are typically administered to a subject (e.g., by direct injection at a tissue site) or generated in situ such that they hybridize with or bind to cellular mRNA and/or genomic DNA encoding a target protein to thereby inhibit expression of the protein, e.g., by inhibiting transcription and/or translation. Alternatively, antisense nucleic acid molecules can be modified to target selected cells and then be administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter can be used.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al., 1987, Nucleic Acids. Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEBS Lett., 215:327-330).

Gene expression of a target protein (e.g., TNFR-2, TNFR-1, or RIP) can be inhibited by targeting nucleotide sequences complementary to the regulatory region of a target gene (e.g., promoters and/or enhancers) to form triple helical structures that prevent transcription of the target gene in a cells. See generally, Helene, C., 1991, Anticancer Drug Des. 6:569-84; Helene, C., 1992, Ann. N. Y. Acad. Sci. 660:27-36; and Maher, 1992, Bioassays 14:807-15. The potential sequences that can be targeted for triple helix formation can be increased by creating a so called “switchback” nucleic acid molecule. Switchback molecules are synthesized in an alternating 5′-3′, 3′-5′ manner, such that they base pair first with one strand of a duplex and then the other, eliminating the necessity for a sizeable stretch of either purines or pyrimidines to be present on one strand of a duplex.

Antisense sequences that decrease expression of TNFR-2 or RIP are useful for, e.g., decreasing programmed necrosis. Sequences that decrease TNFR-1 are useful for, e.g., increasing programmed necrosis.

Ribozymes

Ribozymes are a type of RNA that can be engineered to enzymatically cleave and inactivate other RNA targets in a specific, sequence-dependent fashion. By cleaving the target RNA, ribozymes inhibit translation, thus preventing the expression of the target gene. Ribozymes can be chemically synthesized in the laboratory and structurally modified to increase their stability and catalytic activity using methods known in the art. Alternatively, ribozyme genes can be introduced into cells through gene-delivery mechanisms known in the art. A ribozyme having specificity for a target-encoding nucleic acid (e.g. a TNFR-2, TNFR-1, or RIP MRNA) can include one or more sequences complementary to the nucleotide sequence of the target cDNA, and a sequence having known catalytic sequence responsible for mRNA cleavage (see Cech et al., U.S. Pat. No. 5,093,246 or Haselhoff and Gerlach, 1998, Nature 334:585-591). For example, a derivative of a Tetrahymena L-19 IVS RNA can be constructed in which the nucleotide sequence of the active site is complementary to the nucleotide sequence to be cleaved in a target-encoding MRNA. See, e.g., Cech et al. U.S. Pat. No. 4,987,071; and Cech et al. U.S. Pat. No. 5,116,742. Alternatively, a target mRNA can be used to select a catalytic RNA having a specific ribonuclease activity from a pool of RNA molecules. See, e.g., Bartel, D. and Szostak, J. W., 1993, Science 261:1411-1418.

Ribozymes that cleave TNFR-2 or RIP are useful, e.g., for inhibiting programmed necrosis. On the other hand, ribozyrnes that cleave TNFR-1 are useful, e.g., for increasing programmed necrosis

Cells

Cells used as described herein, for example, in screening assays are those that can undergo programmed necrosis. In general, programmed necrosis is induced in the presence of TNF (e.g., human TNFα), TRAIL, or Fas. The amounts of inducer and conditions for induction are generally the same as those used to induce apoptosis in a cell. Other methods can be used to inhibit the induction of apoptosis and induce programmed necrosis. These include the induction of cells that lack caspase-8 or FADD (e.g., a cell that is genetically engineered to lack normal caspase-8 or FADD expression), contacting the cell with an inhibitor of caspase-8, or contacting the cell with an inhibitor of another molecule in the apoptotic pathway.

The cells can be from a vertebrate such as a mammal, e.g., mouse, rat, guinea pig, monkey, or human.

Inhibition of Caspase-8

In certain assays described herein, it is desirable to inhibit caspase-8 expression or activity. One method of achieving this is to perform screening assays using cells or cell extracts that lack caspase-8 activity. In general, such cells are homozygous for mutant caspase-8 genes (e.g., J3.2 cells). Other methods of inhibiting caspase-8 include introduction of siRNA that targets caspase-8, contacting the cell with a pan-caspase inhibitor (e.g., z-Val-Ala-Asp-fluoromethyl ketone (zVAD-fmk)), or a caspase-8 inhibitor (e.g., z-Ile-Glu-Thr-Asp-fluoromethylketone (z-IETD-fmk)). Methods of detecting caspase-8 are known in the art (e.g., Caspase-8 Detection Kit, Oncogene Research Products, San Diego, Calif.).

Assay of RIP Degradation

Compounds that can modulate the rate or amount of RIP degradation cleavage are useful for modulating programmed necrosis. For example, compounds that increase RIP degradation are useful for decreasing programmed necrosis and inflammatory response. Compounds that decrease RIP degradation are useful for increasing programmed necrosis, e.g., increasing the antiviral response. The invention includes methods of identifying such compounds. The methods include assaying RIP activity in the presence or absence of a test compound. A change in the amount of RIP activity in the presence of the test compound compared to the rate or amount of RIP activity in a corresponding sample that lacks the test compound indicates that the compound is a candidate compound for modulating the rate or amount of RIP activity. The assay can be performed using a cell or in a cell-free sample. In another method, compounds that modulate the rate or amount of RIP degradation can be assayed, e.g., by Western blot detecting RIP, e.g., with an antibody that specifically binds RIP. In one such assay, programmed necrosis is induced in a test sample containing a cell that can undergo programmed necrosis and the test sample is incubated in the presence or absence of a test compound. A difference in the amount of RIP indicates that the test compound can modulate RIP expression. After incubation, Western blotting is performed to detect RIP degradation. A related assay is illustrated in Example 5, infra.

Assay of Recruitment of RIP to TNFR-1

In some embodiments, compounds are identified that modulate the recruitment of RIP to TNFR-1. Generally, assays are conducted to identify compounds that inhibit RIP recruitment to TNFR-1. Such compounds are useful, e.g., for inhibiting programmed necrosis. Recruitment can be assayed by identifying compounds that interfere with the binding of RIP to TNFR-1, e.g., by immunoprecipitation with an antibody that specifically binds RIP or TNFR-1 and detection of RIP-TNFR-1 complexes on Western blots with an antibody that specifically binds TNFR-1 or RIP, respectively. An example of such an assay is provided in Example 7. Other methods such as those described herein that can detect the interaction between two proteins can also be used to detect recruitment. Compounds that interfere with or enhance recruitment are identified by incubating a test compound with a cell that can exhibit programmed necrosis and an inducer of programmed necrosis. The amount of RIP-TNFR-1 binding is detected in the presence or absence of the test compound. A difference in the amount of binding indicates that the test compound is a candidate compound for modulating RIP recruitment to TNFR-1. Such molecules that inhibit recruitment are useful, e.g., for decreasing inflammatory responses during viral infection. Compounds that increase recruitment are useful, e.g., for increasing an anti-viral response by a cell.

Methods of Treatment

The present invention provides for both prophylactic and therapeutic methods of treating a subject at risk of (or susceptible to) a disorder or having a disorder associated with programmed necrosis, e.g., viral infection, or a disorder associated with undesirable inflammation such as rheumatoid arthritis, an inflammatory bowel disease, or septic shock. As used herein, the term “treatment” is defined as the application or administration of a therapeutic agent to a patient or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes the compounds described herein and includes, but is not limited to, small molecules, peptides, peptidomimetics, antibodies, ribozymes, siRNA, and antisense oligonucleotides.

It is possible that some disorders involving programmed necrosis can be caused, at least in part, by an abnormal level of gene product (e.g., TNFR-2, RIP, or TNFR-1) or by the presence of a gene product exhibiting abnormal activity. As such, the reduction in the level and/or activity of such gene products would bring about the amelioration of disorder symptoms.

The,compounds that modulate programmed necrosis that are identified as described herein can be used to treat and/or diagnose a variety of immune disorders. Examples of such disorders or diseases include, but are not limited to, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia; gravis, systemic lupus erythematosis, autoimmune thyroiditis, dermatitis (including atopic dermatitis and eczematous dermatitis), psoriasis, Sjögren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy such as, atopic allergy.

In general, induction or enhancement of programed necrosis is useful for treating infection by a virus that can induce a cell to exhibit programmed necrosis. Such viruses may or may not express an apoptosis inhibitor and include adenovirus, polyoma virus, hepatitis C virus (HCV), HIV-1, Epstein-Barr virus EBV), human herpes virus (HHV)-6/7, HHV-8, bovine herpes virus (BHV)-4, equine herpes virus (EHV)-2, hepatitis B virus (HBV), myxoma virus and poxvirus such as vaccinia virus, and molluscum contagiosum virus (CV).

As discussed herein, successful treatment of disorders associated with programmed necrosis can be brought about by techniques that serve to inhibit the expression or activity of targets such as TNFR-2, RIP degradation, RIP kinase activity, or the recruitment of RIP to TNFR-1. For example, a compound, e.g., an agent identified using an assay described herein, that proves to exhibit negative modulatory activity of TNFR-2, can be used in accordance with the invention to prevent and/or ameliorate symptoms of inflammatory disorders. Such molecules can include, but are not limited to peptides, phosphopeptides, small non-nucleic acid organic molecules, (e.g., anti-sense olignucleotides, ribozymes, or siRNA) or inorganic molecules, or antibodies (including, for example, polyclonal, is monoclonal, humanized, anti-idiotypic, chimeric or single chain antibodies, and Fab, F(ab)₂ and Fab expression library fragments, scFV molecules, and epitope-binding fragments thereof).

Antisense and ribozyme molecules that inhibit expression of a target gene can also be used in accordance with the invention to reduce the level of target gene expression, thus effectively reducing the level of target gene activity. Still further, triple helix molecules can be utilized in reducing the level of target gene activity. Methods of using such molecules are known in the art.

It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target polypeptides exhibiting normal target gene activity can be introduced into cells via gene therapy method.

Another method by which nucleic acid molecules may be utilized in treating or preventing a disease characterized by target expression is through the use of aptamer molecules specific for a target protein. Aptamers are nucleic acid molecules having a tertiary structure which permits them to specifically bind to protein ligands (see, e.g., Osborne, et al., 1997, Curr. Opin. Chem. Biol. 1: 5-9; and Patel, D. J., 1997, Curr. Opin. Chem. Biol. 1:32-46). Since nucleic acid molecules may in many cases be more conveniently introduced into target cells than therapeutic protein molecules may be, aptamers offer a method by which a target protein activity may be specifically decreased without the introduction of drugs or other molecules which may have pluripotent effects.

Antibodies can be generated that are both specific for a target and reduce target activity (e.g., by interfering with the ability of TNFR-2 to participate in programmed necrosis, by modulating the rate or amount of RIP degradation, or by modulating the recruitment of RIP to TNFR-1). Such antibodies may, therefore, be administered in instances where negative modulatory techniques are appropriate for the treatment of disorders related to programmed necrosis. For example, such compounds are useful when it is desirable to decrease programmed necrosis; a compound that increases degradation of RIP, decreases the expression or activity of TNFR-2, or inhibits the recruitment of RIP to NFR-1 will decrease programmed necrosis.

In instances where the target antigen is intracellular and whole antibodies are used, internalizing antibodies may be preferred. Lipofectin or liposomes can be used to deliver the antibody or a fragment of the Fab region that binds to the target antigen into cells. Where fragments of the antibody are used, the smallest inhibitory fragment that binds to the target antigen is preferred. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al., 1993, Proc. Natl. Acad. Sci. USA 90:7889-7893).

The identified compounds that inhibit target gene expression, synthesis and/or activity can be administered to a patient at therapeutically effective doses to prevent, treat, or ameliorate disorders associated with programmed necrosis. A therapeutically effective dose refers to that amount of the compound sufficient to result in amelioration of symptoms of the disorders. Toxicity and therapeutic efficacy of such compounds can be deternined by standard pharmaceutical procedures as described above.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of determination of effective dose for an individual is the ability to directly assay levels of “free” and “bound” compound in the serum of the test subject. Such assays may utilize antibody mimics and/or “biosensors” that have been created through molecular imprinting techniques. The compound which is able to modulate programmed necrosis is used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell, R. J. et al., 1996, Curr. Op. Biotechnol. 7:89-94 and in Shea, K. J., 1994, Trends in Polymer Science 2:166-173. Such “imprinted” affinity matrices are amenable to ligand-binding assays, whereby the immobilized monoclonal antibody component is replaced by an appropriately imprinted matrix. An example of the use of such matrices in this way can be seen in Vlatakis, G. et al (1993) Nature 361:645-647. Through the use of isotope-labeling, the “free” concentration of compound which modulates programmed necrosis can be readily monitored and used in calculations of IC₅₀.

Such “imprinted” affinity matrices can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC₅₀. An rudimentary example of such a “biosensor” is discussed in Kriz, D. et al., 1995, Anal. Chem. 67:2142-2144.

Another method described herein pertains to methods of modulating the expression or activity of a target (e.g., TNFR-2) for therapeutic purposes. Accordingly, in an exemplary embodiment, the modulatory method of the invention involves contacting a cell with a target molecule (e.g., a TNFR-2 or RIP protein or a biologically active fragment thereof) or an agent that modulates one or more of the activities associated with the target.

In one embodiment, the agent stimulates the expression or activity of a target. For example, the agent can stimulate the expression or activity of TNFR-2, thus enhancing programmed necrosis and antiviral activity of the cell. In another example, the agent can stimulate the degradation of RIP, thus decreasing programmed necrosis and decreasing an inflammatory response by the cell. In another embodiment, the agent inhibits one or more activities associated with programmed necrosis as described herein. Examples of such inhibitory agents (e.g., agents that inhibit TNFR-2 expression or activity, RIP degradation, RIP kinase activity, or RIP recruitment to TNFR-2) include antisense nucleic acid molecules, antibodies, and inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted programmed necrosis or in conditions in which it is desirable to increase programmed necrosis (e.g., during viral infection, such as infection by a virus that inhibits cellular apoptosis). In one embodiment, the method involves administering an agent (e.g., an agent identified by a screening assay described herein) or combination of agents that modulates (e.g., up-regulates or down-regulates) target (e.g., TNFR-2 or RIP) expression or activity. In another embodiment, the method involves administering a-target nucleic acid molecule as therapy to compensate for reduced, aberrant, or unwanted target expression or activity.

Stimulation of TNFR-2 or RIP activity is desirable in situations in which TNFR-2 or RIP is abnormally down-regulated and/or in which increased TNFR-2 or RIP activity is likely to have a beneficial effect e.g., during viral infection. For example, stimulation of TNFR-2 activity is desirable in situations in which a TNFR-2 is down-regulated and/or in which increased TNFR-2 activity is likely to have a beneficial effect, e.g., to increase an antiviral response. Likewise, inhibition of TNFR-2 activity is desirable in situations in which TNFR-2 is abnormally up-regulated and/or in which decreased TNFR-2 activity is likely to have a beneficial effect, for example when it is desirable to decrease an inflammatory response such as an inflammatory response caused by vaccination or an inflammatory disorder.

Pharmaceutical Compositions

Compounds that modulate the expression or activity of TNFR-2 (e.g., during programmed necrosis), RIP (e.g., the rate or amount of RIP degradation), or recruitment of RIP to TNFR-1, or other compounds identified as described herein can be incorporated into pharmaceutical compositions. Such compositions typically include the compound and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral (e.g., intravenous, intradermal, subcutaneous), oral, inhalation, transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol, or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes, or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, or sodium chloride in the composition. Prolonged absorption of the injectable compositions can be brought about by including in the composition an agent which delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum-drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically-compatible binding agents, and/or adjuvant materials can be included as part of the composition. The tablets, pills, capsules, troches, and the like can contain any of the following ingredients or compounds of a similar nature: a binder such as microcrystalline cellulose, gum tragacanth, or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide or a nebulizer.

Systemic administration can also be by transmucosal or transdermal means. For transmucosal or transdermal administration, penetrants appropriate to the barrier to be permeated are used in the formulation. Such penetrants are generally known in the art and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in Eppstein et al., U.S. Pat. No. 4,522,811.

It is advantageous to formulate oral or parenteral compositions in dosage unit form for ease of administration and uniformity of dosage. “Dosage unit form” as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g. for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds generally lies within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, about 0.01 to 25 mg/kg body weight, about 0.1 to 20 mg/kg body weight, about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, between 2 to 8 weeks, between about 3 to 7 weeks, or for about 4, 5, or 6 weeks. The skilled artisan will appreciate that certain factors may influence the dosage and timing required to effectively treat a subject, including, but not limited to, the severity of the disease or disorder, previous treatments, the general health and/or age of the subject, and other diseases present. Moreover, treatment of a subject with a therapeutically effective amount of a protein, polypeptide, or antibody can include a single treatment or can include a series of treatments.

For antibodies, the dosage is generally about 0.1 to 20 mg/kg of body weight (generally 1, 3, 5, 8, 15, 18 or 20 mg/kg or more). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. (1997, J. Acquir. Immune Defic. Syndr.s and Human Retrovirol. 14:193).

The present invention encompasses agents that modulate expression or activity. An agent can, for example, be a small molecule. Such small molecules include, but are not limited to, peptides, peptidomimetics (e.g., peptoids), amino acids, amino acid analogs, polynucleotides (e.g., siRNA or antisense RNA), polynucleotide analogs, nucleotides, nucleotide analogs, non-nucleic acid organic compounds and inorganic compounds (i.e.,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, sex, and diet of the subject, the time of administration, the route of administration, the rate of excretion, any drug combination, and the degree of expression or activity to be modulated.

A nucleic acid molecule that modulates TNFR-2 expression or activity or exhibits one of the other desired activities described herein can be inserted into vectors and used as gene therapy vectors. Gene therapy vectors can be delivered to a subject by, for example, intravenous injection, local administration (see Nabel et al., U.S. Pat. No. 5,328,470) or by stereotactic injection (see e.g., Chen et al., 1994, Proc. Natl. Acad. Sci. USA 91:3054-3057). The pharmaceutical preparation of the gene therapy vector can include the gene therapy vector in an acceptable diluent or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

A compound as described herein can be used for the preparation of a medicament for use in any of the methods of treatment described herein.

### EXAMPLES

### Example 1

### Materials and Methods

### Reagents and Cell Lines

Jurkat cells deficient in FADD or caspase-8 were prepared as described in Curr. Biol., 1998, 8:1001; Cell Growth Differ., 1999, 10:797), RIP -/- Jurkat cells were obtained from B. Seed (EMBO J., 1996, 15:6189); TNFR-2/p80 overexpressing cells were generated by stable transfection of Jurkat cells and selected for G418 resistance. The RIP-K45A and RIP-D324A mutations were generated by Quikchange PCR mutagenesis (Stratagene, Calif.) and cloned into pEGFP-N1 (BD Clontech, CA) at NheI and BamiHI. RIPn (amino acids 1-324) and RIPc (amino acids 325-671) were generated by PCR cloning and inserted at XhoI and EcoRI sites into pEGFP-N1 and pEGFP-C1, respectively. The cDNAs for E8, K13 and cFLIPs were cloned into pEGFP-Ni at XhoI and EcoRI sites (for E8 and K13). The MC159 plasmid has been described. The expression plasmid for ASK1 was provided by Dr. Ichiro (Science, 1997, 275:90-94). Anti-caspase-8 monoclonal antibody (C15) was provided by P. Krammer (EMBO J., 1995, 14:5579-5588). Other antibodies used in this study include anti -TRADD, -FADD, -RIP (BD Transduction lab, CA), -TRAF2 (Santa Cruz Biotechnology, CA), -Fas (Kamiya, CA), -TNFR-1 (R&D Systems), -TNFR-2 (R&D Systems), and -HA (Roche, Ind.). The anti-TNFR-1 antibody used in Western blots has been described. Z-Val-Ala-Asp-fluoromethyl ketone (zVAD-fmk) and geldanamycin (GA) were purchased from Biomol (PA) and Sigma (MO), respectively.

### Immunoprecipitation

50 million Jurkat cells were stimulated with 50 ng/ml of recombinant human tumor necrosis factor a (rhTNFα) for the indicated amount of time. In some experiments, cells were pre-stimulated with agonist TNFR-2 antibodies for six hours prior to stimulation with TNF to induce TRAF2 degradation (Chan et al., 2000, Eur. J. Immunol. 30:652-660). Cells were quenched and washed in cold PBS before lysis in immunoprecipitation (IP) buffer (150 mM NaCl; 20 mM Tris. Cl [pH 7.5]; 1% Nonidet P-40; 1 mM EDTA; 3 mM NaF; 1 mM β-glycerophosphate; 1 mM sodium orthovanadate; 1× Complete protease inhibitor cocktail (Roche, Ind.)). Lysates were pre-cleared with three washes of protein G agarose beads (15 minutes incubation at 4° C. for each wash) and then immunoprecipitated with 10 μg of goat anti-human TNFR-1 polyclonal antibody (R&D Systems, MN) and protein G agarose beads for two to three hours at 4° C. Immune complexes were washed 2× with IP buffer, 2× with high salt IP buffer (500 mM NaCl) and 1× with IP buffer. Inmunoprecipitations were resolved on 10% Bis/Tris NuPAGE gels (Invitrogen, Calif.) and transferred to nitrocellulose membranes for standard Western blot analysis. For 293T transfections and co-immunoprecipitation, cells were transfected with FuGENE™ 6as per manufacturer's instructions (Roche, Ind.). Twenty-four hours later, cells were lysed in IP buffer and immunoprecipitation was performed using anti-RIP (BD Transduction lab, CA) or anti-TNFR-1 (R&D Systems, MN) antibodies as indicated.

TNF Cytotoxic Assay

Jurkat cells were incubated with the indicated doses of rhTNFα (R&D Systems, MN) for 8-24 hours. Cell viability was determined by exclusion of propidium iodide (P) using the FACScan flow cytometry (BD Bioscience, CA). Data were analyzed using FlowJo (Treestar Inc., CA). All samples were collected on constant time and the percent cell loss was calculated with the equation:

% Cell Loss=(1−(number of live cells in treated sample/number of live cells in untreated sample))×100%. In some cases, cells were pre-incubated with geldanamycin for 12 hours or zVAD-fmk for 30 minutes prior to stimulation with rhTNFα.

In Vitro VV Infections

Jurkat cell derivatives were infected with 20 plaque-forming units (pfu) of virus per cell at 37° C. in medium containing 2.5% fetal bovine serum (FBS). For all infections, cells were incubated for one hour with virus before changing media. At six hours after infection, cells were incubated in fresh medium with or without rhTNFα at the indicated concentrations. Twelve hours later, cell death was detected by forward/side scatter or by PI exclusion on FACS. Apoptosis was detected by TUNEL staining using the In Situ Cell Death Detection Kit (Roche, Ind.). Fixed cells were incubated with a monoclonal antibody to an early protein of W virus (E3L) to monitor infectivity, followed by incubation with a CyS-conjugated anti-mouse antibody (Jackson Immunoresearch, PA). Using FACSCalibur (BD Bioscience, CA) for analysis, only individual cells positive for E3L staining were analyzed for cell death.

In Vivo VV Infection

C57BL/6 mice and TNFR-2 -/-mice in the C57BL/6 background (8-10 weeks old) were infected via intraperitoneal administration of 10⁶ pfu of wild-type vaccinia virus (VV). Four days after infection, fat pads, spleens and livers were harvested from VV infected mice. Tissues were divided into two portions. Half of the tissues were fixed in 10% formalin for histological analysis. Paraffin embedded tissues were sectioned at 4 mmand stained with hematoxylin and eosin (H&E). The remaining tissues were homogenized in 1-2 ml of medium for determination of viral titers. Ten-fold serial dilutions were made and overlaid on Vero cells. After a 90 minute incubation at 37° C., the cells were overlaid with a 0.4% agarose solution in EMEM (Minimal Essential Medium with Earle's salts). On day two, the plates were stained with neutral red. Plaques were counted on day three.

### Example 2

### TNFR-2 Facilitates Caspase-Independent Proprammed Necrosis in the Absence of FADD and Caspase-8

TNFR-2 can facilitate TNFR-1-mediated apoptosis (Chan et al., 2000, Eur. J. Immunol. 30:652-660). To examine whether TNFR-2 signaling may also potentiate TNF-induced programmed necrosis, TNFR-2 was introduced into FADD -/- or caspase-8 -/- Jurkat cells.

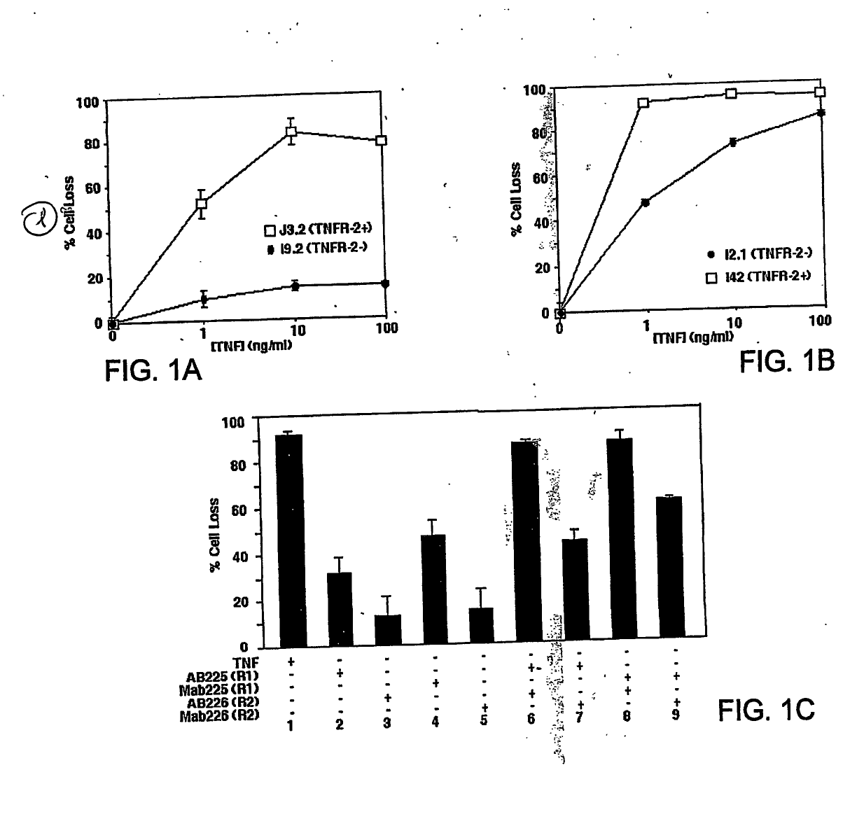

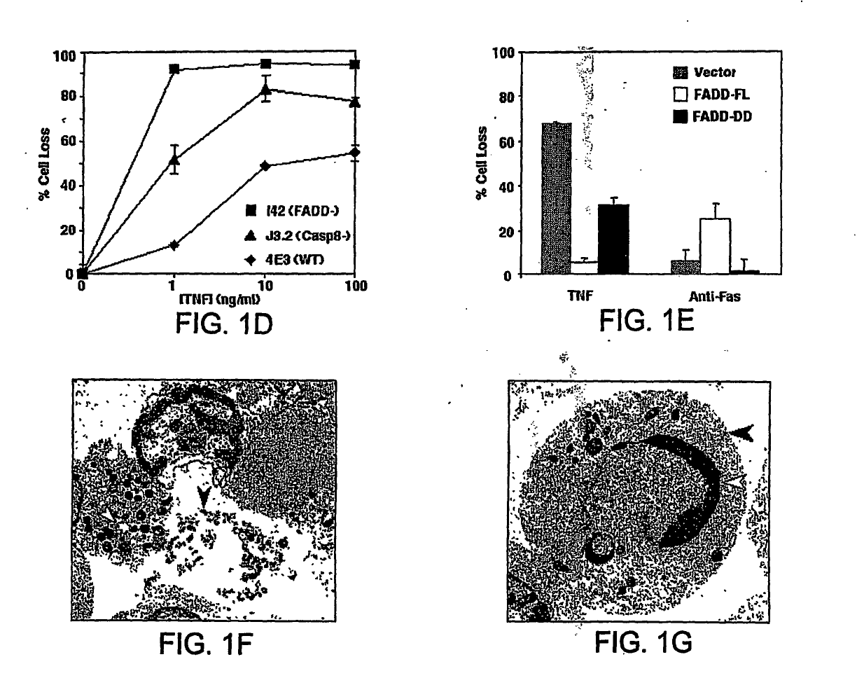

Although caspase-8 -/- cells (I9.2 cells) are normally resistant to TNF-induced death (Juo et al., 1998, Curr. Biol. 8:1001-1008), the introduction of TNFR-2 (J3.2 cells) led to a striking sensitivity to TNF-induced cytotoxicity (FIG. 1A). In a similar fashion, the introduction of TNFR-2 into FADD -/- Jurkat cells also led to an enhancement in TNF-induced cytotoxicity, although FADD -/- cells also underwent TNF-induced death at a lower level in the absence of TNFR-2 (FIG. 1B; Holler et al., 2000, Nat. Immunol. 1:489-495; Khwaja et al., 1999, J. Biol. Chem. 274:36817-36823).

The cytotoxic effects of TNF in J3.2 cells could be mimicked by using a combination of agonistic antibodies against TNFR-1 (AB225 and Mab225) and TNFR-2 (AB226; FIG. 1C, compare lanes 1, 6, and 8). However, either antibody alone was much less effective in triggering cell death compared with TNF (FIG. 1C, compare lanes 1-4). When used alone, greater death was obtained with two TNFR-1 specific antibodies compared to the TNFR-2 antibodies (FIG. 1C, compare lanes 2-5). Moreover, a TNFR-2 specific antagonistic antibody (Mab226) failed to synergize with either TNFR-1 antibodies (FIG. 1C, compare lanes 6-7 and 8-9).

The antibody data and the data showing that TNF can induce programmed necrosis in TNFR-2 negative FADD -/- cells demonstrate the novel finding that programmed necrosis is triggered through TNFR-1 but requires an enhancing signal from TNFR-2.

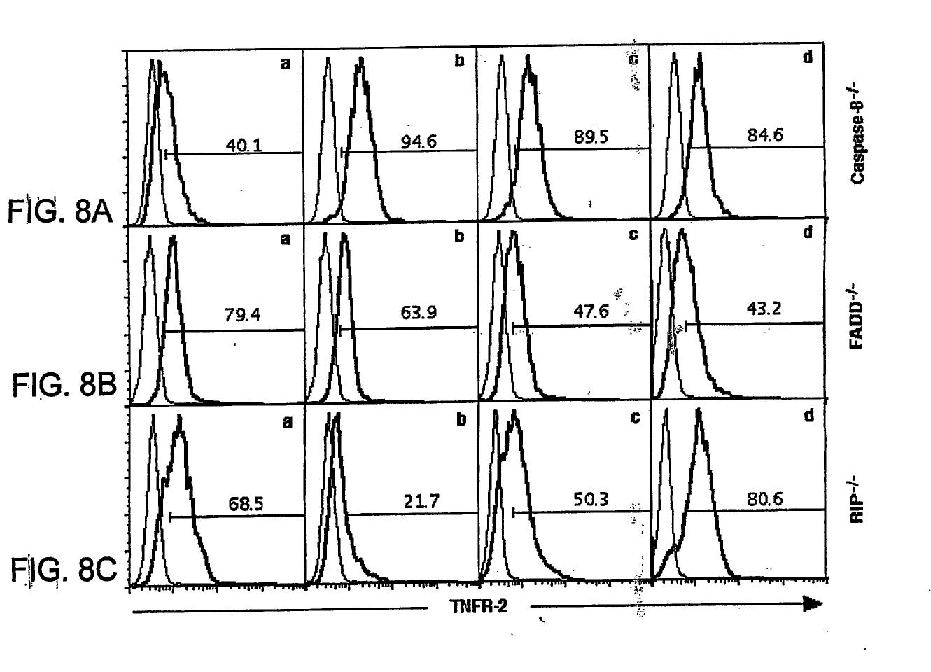

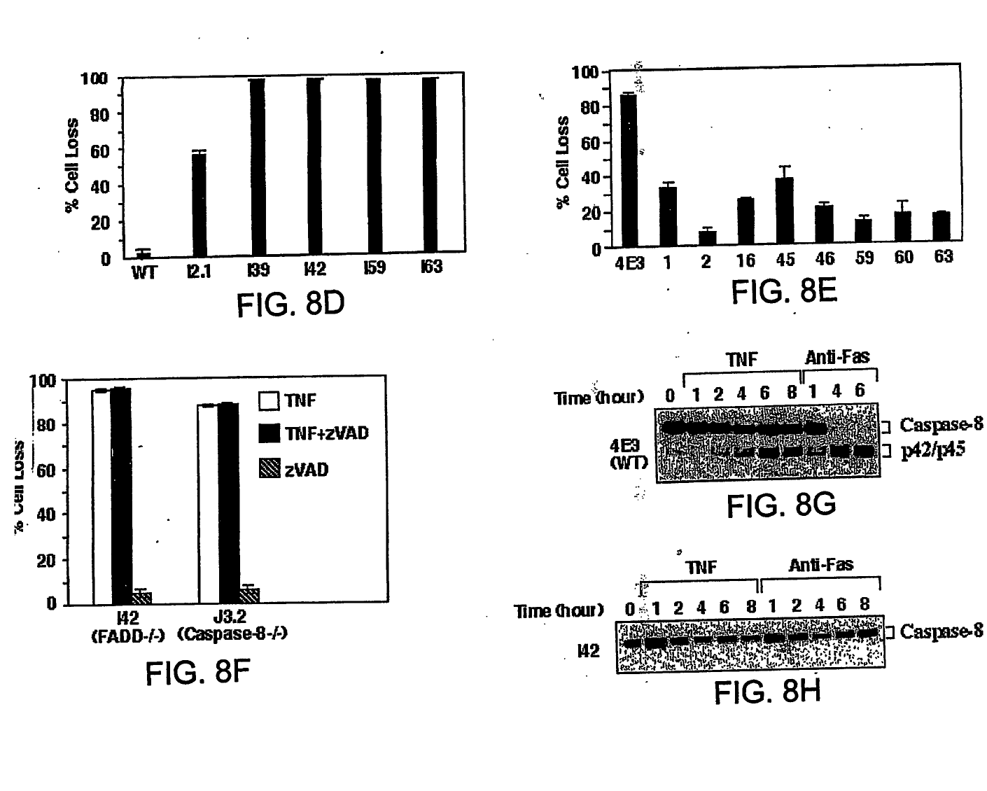

When the cytotoxic responses of TNFR-2-expressing wild-type (4E3), caspase-8 -/- (J3.2) and FADD -/- (I42) cells were compared, it was clear that the loss of caspase-8 and FADD dramatically enhanced cell death responses to TNF (FIG. 1D). The enhanced response to TNF was not due to cell line idiosyncrasies as multiple TNFR-2 positive clones of each variety exhibited consistent phenotypes (FIG. 8A-8E). Moreover, the reconstitution of full-length FADD in FADD -/-cells resulted in the suppression of TNF-induced programmed necrosis (FIG. IE). Unlike classical Fas-induced apoptosis, TNFa-induced programmed necrosis in caspase-8 -/-J3.2 was distinguished by extensive loss of membrane integrity, swelling of intracellular organelles, and limited chromatin condensation (FIG. 1F-G).

### Example 3

### TNFR-2-Facilitated Programmed Necrosis Requires the Protein Kinase RIP

Holler et. al. have shown that the protein kinase RIP is essential for Fas-induced programmed necrosis (Holler et al., 2000, Nat. Immunol. 1:489-495). Although the major function of RIP appears to be the induction of NF-KB on TNF stimulation (Kelliher et al., 1998, Immunity 8:297-303), RIP over-expression can also cause spontaneous cell death under certain circumstances (Stanger et al., 1995, Cell 81:513-523).

To investigate whether RIP is also involved in TNFR-2 facilitated programmed necrosis, TNFR-2 was introduced into RIP -/- Jurkat cells (Ting et al., 1996, EMBO J. 15:6189-6196). As previously reported, TNFR-2 expression in RIP -/- cells (R1.1 cells) failed to enhance TNF-induced death over the level observed in the parental cells (FIG. 8E and Pimentel-Muinos et al., 1999, Immunity 11:783-793).

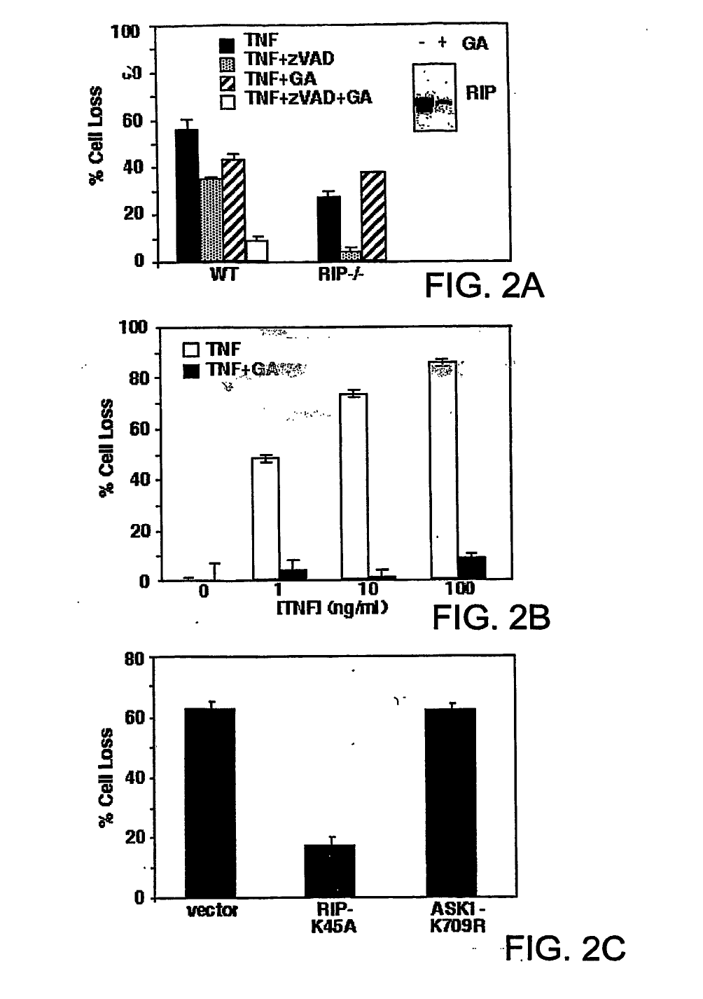

Nevertheless, TNF-induced death was completely inhibited in TNFR-2+RIP -/-Jurkat cells by the pan-caspase inhibitor zVAD-fink (FIG. 2A). This is in stark contrast to the caspase-8 -/- and FADD -/-cells, in which zVAD-fmk failed to provide any protection against TNF-induced programmed necrosis (FIG. 8F). In wild-type 4E3 cells, zVAD-fmk had a partial protective effect, indicating that 4E3 cells died by apoptosis but a significant fraction of cells underwent programmed necrosis when caspases were suppressed. Treatment with geldanamycin (GA), which targets Hsp90 and indirectly reduces RIP protein expression (FIG. 2A, inset and Lewis et al., 2000, J. Biol. Chem. 275:10519-10526), also modestly decreased TNF-induced death in 4E3 cells. However, zVAD-fmk and geldanamycin (GA) synergized to completely suppress TNF-induced death in wild-type 4E3 cells (FIG. 2A). The addition of zVAD-fmk or geldanamycin (GA) alone had no effect on cell viability. Significantly, GA treatment completely suppressed TNF-induced programmed necrosis in caspase-8 -/- J3.2 and FADD -/- I42 cells, but had no effect on TNF-induced apoptosis in RIP -/- cells (FIGS. 2A-2B and data not shown). Analysis of RIP protein expression on Western blot showed >90% reduction in RIP protein level in cells treated with GA. Furthermore, a kinase-inactive RIP, but not a kinase-inactive form of apoptosis signaling kinase ASK1, strongly suppressed TNF-induced programmed necrosis in J3.2 cells (FIG. 2C). Caspase-8 cleavage was not observed in I42 or J3.2 cells (FIGS. 8G-8H).

Altogether, these data demonstrate that two programmed cell death pathways, caspase-dependent apoptosis and RIP-mediated programmed necrosis, are functional in wild-type 4E3 cells.

### Example 4

### TNFR-2 Facilitates Programmed Necrosis by Enhancing RIP Recruitment to TNFR-1

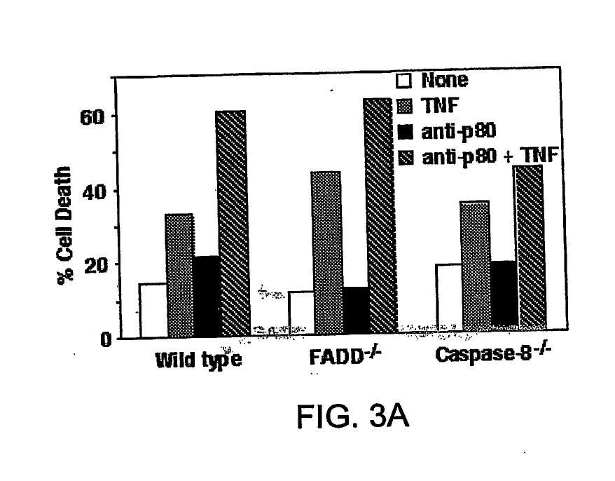

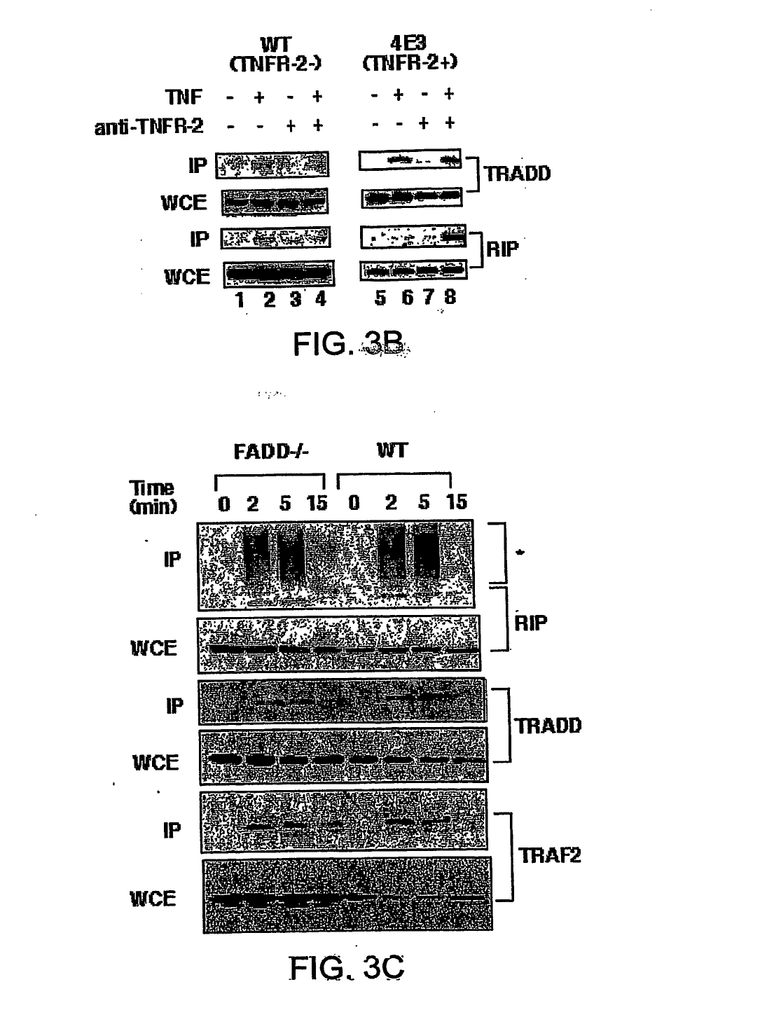

The mechanism by which TNFR-2 facilitates programmed necrosis was examined. Incubating TNFR-2+wild-type, FADD -/- and caspase-8 -/- cells with an agonistic TNFR-2 antibody enhanced subsequent cell death induced by TNF (FIG. 3A). Because TNFR-2 signaling causes degradation of TRAF2 (Chan et al., 2000, Eur. J. Immunol. 30:652-660), a factor that binds to both TNFR-1 and TNFR-2, it is possible that TNFR-2-mediated degradation of TRAF2 might reduce steric hindrance and enhance RIP recruitment to the activated TNFR-1 complex (Wajant et al., 2001, Int. J. Biochlem. Cell Biol. 33:19-32). Indeed, when cells were re-activated through TNFR-2 to induce TRAF2 degradation prior to stimulation with TNF, RIP recruitment to TNFR-1 was dramatically enhanced (FIG. 3B, compare lanes 6 and 8). By contrast, the mere absence of FADD had no effect on the recruitment of RIP, TRADD, or TRAF2 to the activated TNFR-1 complex (FIG. 3C).

These results therefore show that TNFR-2 signaling facilitates programmed necrosis in large part by enhancing RIP recruitment to the activated TNFR-1 complex. Therefore, inhibiting TNFR-2 signaling can decrease recruitment of RIP to TNFR-1 complex and can inhibit programmed necrosis and ameliorate the effects of programmed necrosis, e.g., an inflammatory response. In addition, enhancement of TNFR-2 signaling can increase recruitment of RIP to TNFR-1 and therefore increase programmed necrosis, e.g., to increase an anti-viral response by the cell.

These data also demonstrate that TRAF2 degradation can be used to assay activation of programmed necrosis, e.g., by assaying TRAF2 degradation as described above.

### Example 5

### Caspase-Mediated Cleavage and Inactivation of RIP During Apoptosis Dampens Programmed Necrosis

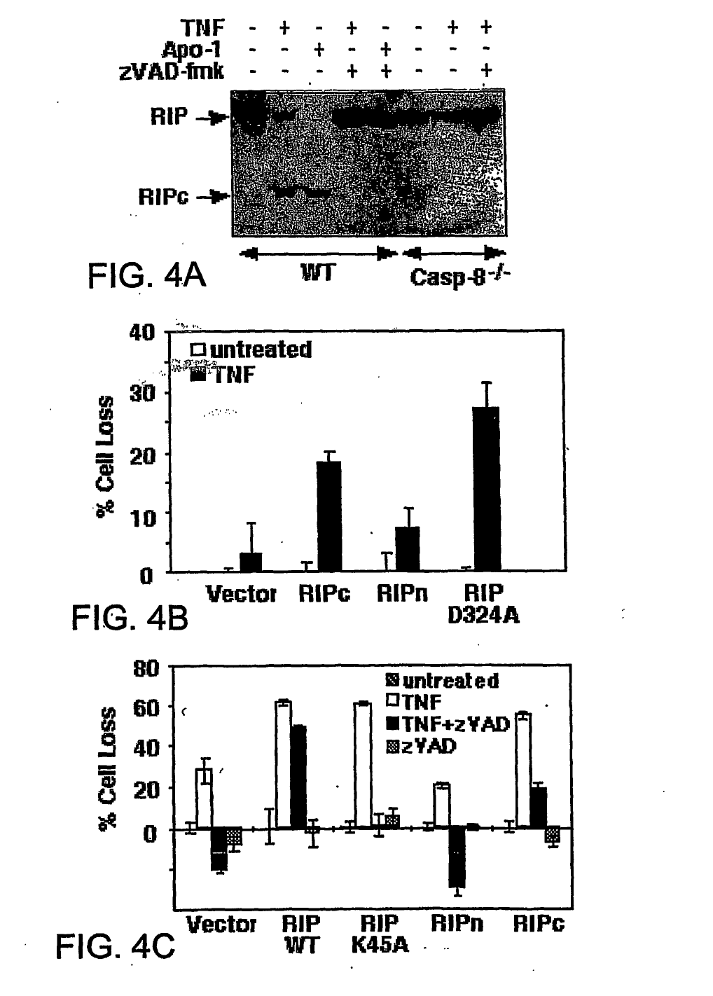

Although RIP recruitment to TNFR-1 was enhanced when TNFR-2 was pre-activated, apoptosis, but not programmed necrosis, was still the dominant response in wild-type 4E3 cells (FIG. 2A and Chan et al., 2000, Eur. J. Immunol. 30:652-660). Genetic ablation of the apoptotic pathway in FADD -/- and caspase-8 -/- cells or inhibition of caspases by tetrapeptide inhibitors caused increased programmed necrosis; leading to the question of whether caspase-8 activation during apoptosis inactivates the necrotic pathway by proteolytic cleavage of RIP. Indeed, RIP was cleaved in TNF or anti-Fas stimulated wild-type 4E3 cells, but not in either caspase-8 -/- (FIG. 4A) or FADD -/- cells. Moreover, RIP cleavage was potently inhibited by zVAD-fmk.

To investigate whether RIP cleavage by-caspases abrogates its ability to induce programmed necrosis, we reconstituted the TNFR-2+RIP -/- cells with various RIP mutants. The N-terminal cleavage product of RIP (RIPn) failed to restore response to TNF (FIGS. 4B-C). However, both the wild-type RIP and the non-cleavable mutant RIP-D324A potently restored TNF killing in RIP -/- cells (FIGS. 4B-C and Pimentel-Muinos et al., 1999, Immunity 11:783-793). Furthermore, TNF-induced cell death in wild-type RIP-reconstituted cells was not blocked by caspase inhibitors (FIG. 4C). Although the kinase inactive (K45A) and C-terminal fragment of RIP (RIPc) also restored killing by TNF, it was potently inhibited by zVAD-fmk, suggesting that apoptosis, but not programmed necrosis, was restored (FIG. 4C). Collectively, these data showed that RIP is cleaved and inactivated by caspase-8 during apoptosis. Thus, in addition to TNFR-2 signaling, caspase-8 inhibition is also required to switch on the programmed necrosis machinery.

These data demonstrate that both TNFR-2 and caspase-8 can serve as targets for compounds to modulate necrotic cell death.

### Example 6

### Programmed Necrosis is Required for TNF-Mediated Killing of Vaccinia Virus-Infected Cells in Vitro

One possible scenario where caspase activity can be inhibited is during viral infections. Many viruses encode gene products that specifically inhibit apoptosis. This is believed to be important for the successful propagation of the virus within the host by delaying the demise of the infected cell. However, the programmed necrosis described herein could eliminate infected cells when apoptosis is suppressed.

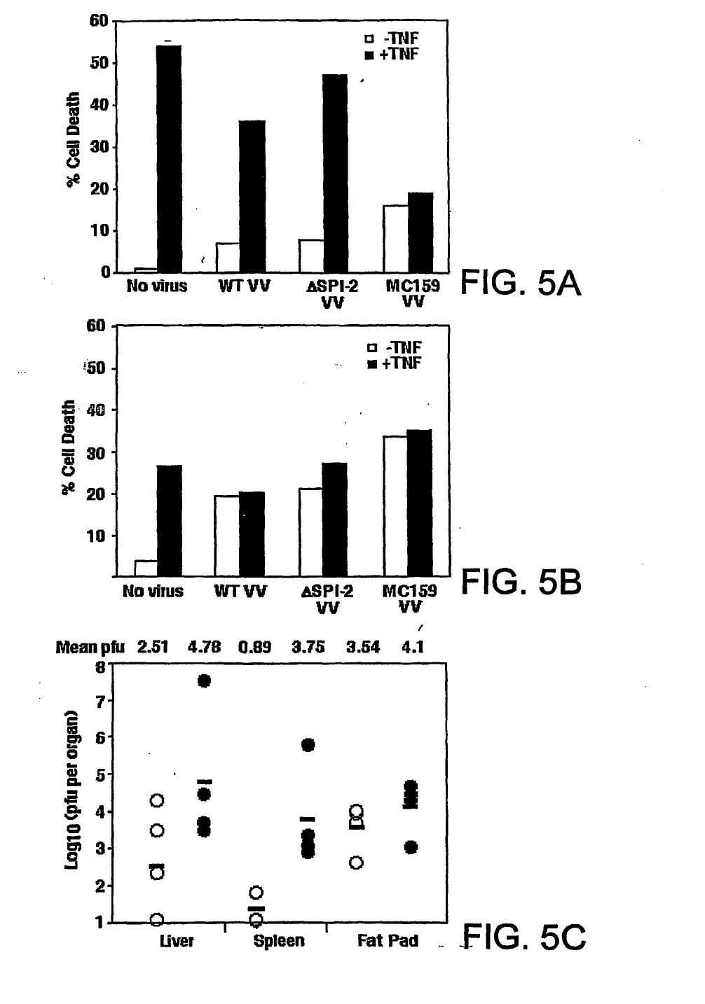

To investigate the potential role of TNF-induced programmed necrosis in an anti-viral response, various Jurkat cell lines were infected with recombinant vaccinia virus (VV). VV encodes a gene, SPI-2, that is homologous to the cytokine response modifier A (crmA) gene found in cowpox virus and is a potent inhibitor of caspase-8 and TNF- or Fas-induced apoptosis (Zhou et al., 1997, J. Biol. Chem. 272:7797-7800). However, TNFR-2+ caspase-8 -/- cells remained responsive to TNF-induced death upon VV infection, albeit at a lower level than uninfected controls (FIG. 5A). Infection of VV with a specific deletion of SPI-2 modestly increased the cell death response to TNF. Strikingly, replacing SPI-2 with the apoptosis inhibitor MC159 resulted in a complete abrogation of TNF-induced cell death in the infected cells (FIG. 5A). Similar results were obtained with wild-type and FADD -/- Jurkat cells. Importantly, the majority of the dying cells were not positive for the apoptosis marker TONEL, implying that TNF-induced death in VV-infected Jurkat cells was necrotic rather than apoptotic. In contrast to infection in wild-type, caspase-8 -/- or FADD -/- cells, RIP -/- cells infected with VV were resistant to TNF-induced death (FIG. 5B). The deletion of SPI-2 had no significant effect on TNF-induced death FIG. 5B). Thus, it can be concluded that RIP-mediated programmed necrosis is crucial for TNF-induced cytotoxicity in VV-infected cells.

TNF is one of the major cytokines produced during viral infections. Hence, programmed necrosis is likely to play a general role in anti-viral responses, especially in response to infection by cytopathic viruses and poxviruses.

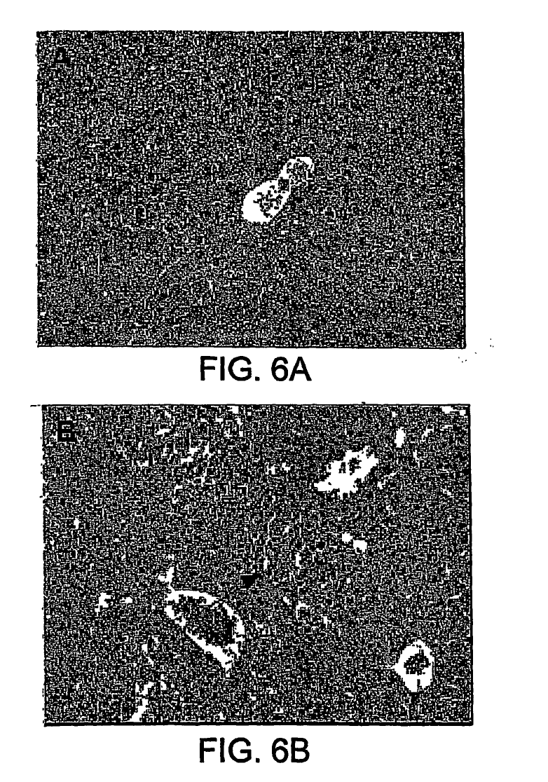

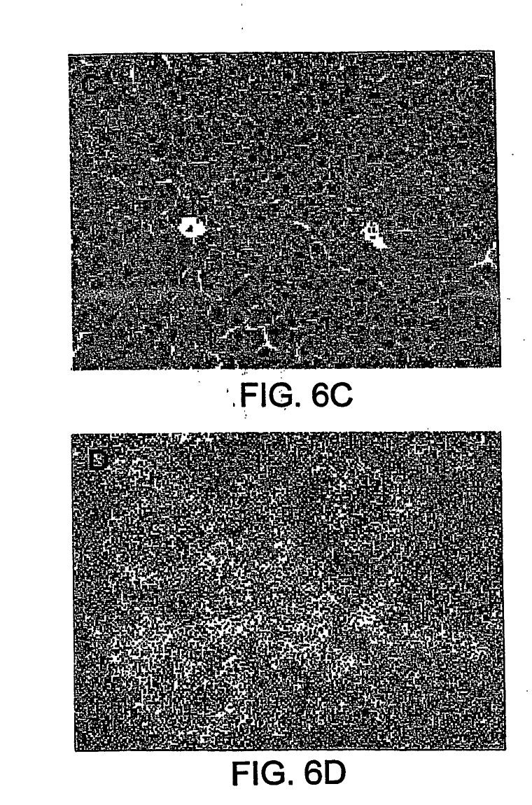

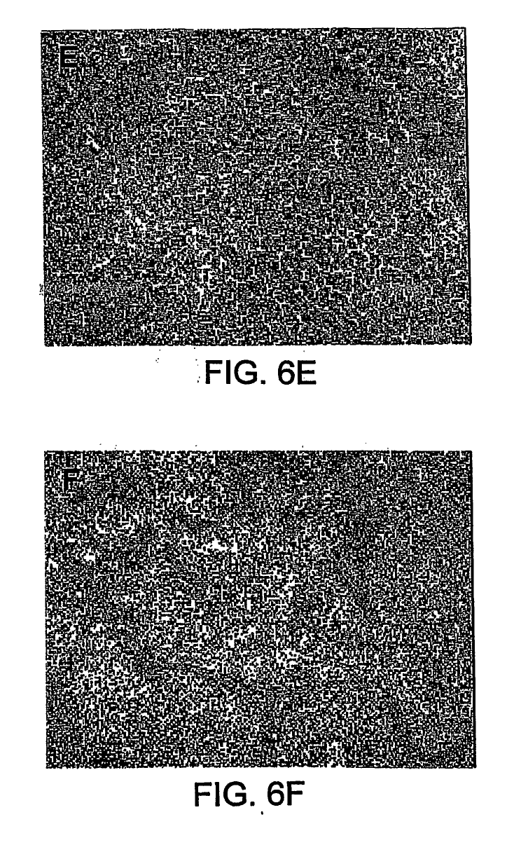

Because TNF-induced programmed necrosis in cultured cells requires TNFR-2, the potential role of TNFR-2 in controlling VV infection in vivo was tested. TNFR-2 -/-mice and control C57BL/6 mice were infected with VV and viral titers in different organs were determined four days post-infection. Higher viral titers were recovered in the fat pads (three-fold), the livers (100-fold) and especially in the spleens (1000-fold) in the TNFR-2 -/-mice compared with the wild-type mice (FIG. 5C). Examination of the infected tissues revealed a large number of inflammatory foci in the liver of the wild-type infected mice, but not in control uninfected mice or TNFR-2 -/-infected mice (FIGS. 6A-C). Minimal tissue abnormalities are seen in the liver (FIG. 6A) and spleen (FIG. 6D) of uninfected wild-type C57BL/6 mice. By contrast, VV infection produced abundant foci of hepatic mononuclear cell inflammation (arrow) in VV-infected C57BL/6 mice (FIG. 6B), but not in the VV-infected TNFR2 -/- mice (FIG. 6C). Prominent disorganization and reduced cell density were observed in the follicles of the spleens of VV-infected TNFR2 -/- mice (FIG. 6F), but not in infected C57BL/6 mice (FIG. 6E). In addition, there was a general dissolution of follicle structures in the spleen of the infected TNFR-2 -/- mice (FIGS. 6F). These data are consistent with the hypotheses that TNFR-2 facilitates programmed necrosis of the infected tissues and triggers an inflammatory reaction that is crucial for the subsequent initiation of adaptive immunity against VV infection.

These data demonstrate that programmed necrosis is required for TNF-mediated killing of virally-infected cells. It also showns that this process requires RIP.

### Example 7

### Viral Inhibitors of Progammed Necrosis

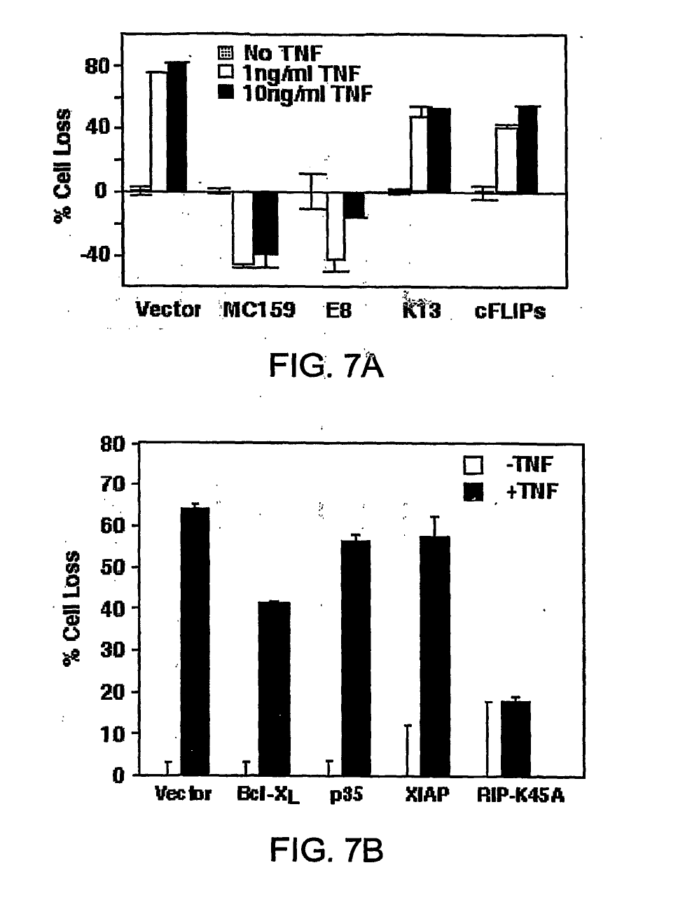

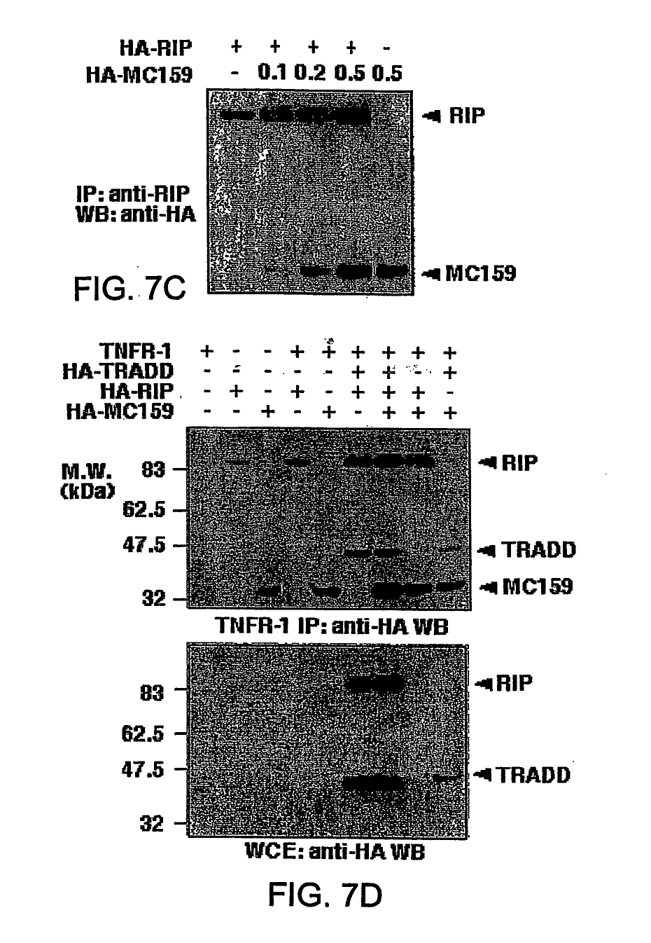

Because many viruses have specific gene products that suppress apoptosis and there is a strong selective advantage to blocking host cell death, it was hypothesized that certain viral gene products may also inhibit programmed necrosis. Indeed, transient expression of MC159 led to strong inhibition of TNF-induced death in caspase-8 -/- J3.2 cells (FIG. 7A), consistent with its inhibitory effect in VV infection (FIG. 5A). MC159 is an anti-apoptotic gene from the poxvirus, Molluscum contagiosum (MCV) that shares sequence homology with the death effector domains (DEDs) of caspase-8 and caspase- 10 but has no enzyme domain. Because of their homology to caspase-8 or FLICE and their ability to inhibit its enzyme function and apoptosis, MC159 and other homologous viral inhibitors of apoptosis are sometimes referred to as vFLIPs (viral FLICE-like inhibitor proteins; Thome et al., 1997, Nature 386:517-521; Senkevich et al., 1996, Science 273:813-816; Hu et al., 1997, J. Biol. Chem. 272:9621-9624; Bertin et al., 1997, Proc. Natl. Acad. Sci. USA 94:1172-1176).

It was also found that other DED-containing molecules, such as the equine herpesvirus-2 (EHV-2) E8, the Kaposi sarcoma-associated herpesvirus (KSHV/HHV-8) K13, and the cellular FLIP (cFLIP), also protected cells from programmed necrosis, although the protection by K13 and CFLIP was weaker (FIG. 7A). By contrast, other known apoptosis inhibitors such as p35 and XLAP failed to confer protection. Bcl-X_{L} appeared to have a moderate suppressive effect on TNF-induced programmed necrosis (FIG. 7B). Collectively, these data showed that the ability to suppress programmed necrosis is not a common property of all apoptosis inhibitors and is specific to only a subset of DED-containing molecules including MC159, E8, K13, and cFLIP.

Because RIP is essential for initiating programmed necrosis, it was postulated that MC159 and other DED-containing molecules might interfere with programmed necrosis by binding to RIP and preventing RIP recruitment to TNFR-1. As previously reported (Chaudhary et al., 1999, Oncogene 18:5738-5746), RIP and MC159 did interact strongly (FIG. 7C). However, MC159 failed to interfere with the binding of RIP to the TNFR-1 signaling complex (FIG. 7D). In fact, the association between MC159 and RIP was abrogated when RIP was bound to the TNFR-1 complex (FIG. 7D). These results showed that the target of MC159 inhibition during programmed necrosis is likely to be further downstream from RIP.

The finding that DED-containing molecules are involved in programmed necrosis and can suppress programmed necrosis demonstrates that some viruses can inhibit programmed necrosis. Agents that decrease the inhibitory activity of such molecules can increase programmed necrosis. Also, since cFLIP has a moderate inhibitory effect on programmed necrosis, drugs that target CFLIP will increase or decrease programmed necrosis and can be used to modulate viral infection.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of determining whether a test compound can modulate programmed necrosis, the method comprising
(a) providing a test cell capable of undergoing programmed necrosis;
(b) contacting the test cell with an inducer of programmed necrosis and a test compound, thereby providing a test sample; and
(c) determining the effect of the test compound on TNF Receptor (TNFR)-2 expression or activity, Receptor-Interacting Protein (RIP) degradation, RIP recruitment to Tumor Necrosis Factor Receptor (TNFR-1), or TNFR-Associated Factor 2 (TRAF2) degradation; wherein a change in the effect in the test sample compared to the effect in a test cell contacted with an inducer of programmed necrosis in the absence of the test compound indicates that the test compound can modulate programmed necrosis.

2. The method of claim 1, wherein the inducer of programmed necrosis is a Tumor Necrosis Factor (TNF), a TNF-related apoptosis-inducing ligand (TRAIL), or a Fas ligand.

3. The method of claim 1, wherein the test compound increases programmed necrosis.

4. The method of claim 1, wherein the test compound decreases programmed necrosis.

5. The method of claim 1, wherein the test compound affects TNFR-2 expression or activity.

6. The method of claim 1, wherein the test compound affects RIP degradation.

7. The method of claim 1, wherein the test compound affects RIP recruitment to TNFR-1.

8. The method of claim 1, wherein the test compound affects TRAF2 degradation.

9. The method of claim 1, wherein the cell contains a recombinant TNFR-2.

10. The method of claim 1, wherein the test compound specifically binds to TNFR-2.

11. The method of claim 1, wherein the test compound is an antibody or fragment thereof.

12. The method of claim 1, further comprising inhibiting caspase-8 expression or activity in the test cell.

13. The method of claim 1, wherein the cell is caspase 8 -/-.

14. The method of claim 1, wherein the test compound specifically binds to RIP.

15. A method of modulating programmed necrosis in a cell, the method comprising
(a) providing a cell that can undergo programmed necrosis and is optionally exposed to an inducer of programmed necrosis; and
(b) contacting the cell with a compound that can modulate TNF Receptor (TNFR)-2 expression or activity, Receptor-Interacting Protein (RIP) degradation, RIP recruitment to Tumor Necrosis Factor Receptor (TNFR-1), or TNFR-Associated Factor 2 (TRAF2) degradation, thereby modulating programmed necrosis.

16. The method of claim 15, wherein the inducer of programmed necrosis is a Tumor Necrosis Factor (TNF), a TNF-related apoptosis-inducing ligand (TRAIL), or a Fas ligand.

17. The method of claim 15, wherein the compound increases programmed necrosis.

18. The method of claim 15, wherein the compound decreases programmed necrosis.

19. The method of claim 15, wherein the compound modulates TNFR-2 expression or activity.

20. The method of claim 15, wherein the compound modulates RIP degradation.

21. The method of claim 15, wherein the compound modulates RIP recruitment to TNFR-1.

22. The method of claim 15, wherein the compound modulates TRAF2 degradation.

23. The method of claim 15, wherein the cell contains a recombinant TNFR-2.

24. The method of claim 15, further comprising contacting the cell with a compound that modulates the expression or activity of caspase-8.

25. The method of claim 15, wherein the compound specifically binds to TNFR-2.

26. The method of claim 15, wherein the compound is an antibody.

27. The method of claim 15, wherein the cell is contacted with a compound that specifically binds to RIP.

28. The method of claim 15, wherein the cell is within a subject.

29. The method of claim 15, wherein the cell is a cultured cell.

30. The method of claim 15, wherein the compound decreases degradation of RIP, thereby increasing programmed necrosis.

31. The method of claim 15, further comprising contacting the cell with a second compound that can induce programmed necrosis.

32. The method of claim 31, wherein the inducer of programmed necrosis is a Tumor Necrosis Factor (TNF), a TNF-related apoptosis-inducing ligand (TRAIL), or a Fas ligand.

33. The method of claim 15, wherein the compound increases degradation of RIP, thereby decreasing programmed necrosis.

34. The method of claim 15, wherein the compound increases recruitment of RIP to TNFR-1, thereby increasing programmed necrosis.

35. The method of claim 34, further comprising contacting the cell with a compound that induces programmed necrosis.

36. The method of claim 35, wherein the inducer of programmed necrosis is a Tumor Necrosis Factor (TNF), a TNF-related apoptosis-inducing ligand (TRAIL), or a Fas ligand.

37.-46. (canceled)
